(19) United States
(12) Patent Application Publication
(10) Pub. No.: US 2005/0152835 A1
(43) Pub. Date: Jul. 14, 2005

(54) Technetium-99M glucarate methods of use for monitoring tissues

(76) Inventors: Koon Yan Pak, Malvern, PA (US); Giuliano Mariani, Pisa (IT); Zhonglin Liu, Oro Valley, AZ (US)

Correspondence Address: Pepper Hamilton LLP;Firm 21269, One Mellon Center, 50th Floor, 500 Grant Street, Pittsburgh, PA, 15219, US

(21) Appl. No.: 10/978,929

(22) Filed: Nov. 1, 2004

Related U.S. Application Data
(60) Provisional application No. 60/516,081, filed on Oct. 31, 2003.

(51) Int. Cl. A61K 51/00

(52) U.S. Cl. 424/1.11

(57) ABSTRACT

A method of characterizing a resistant tissue includes imaging ^{99m}Tc-GLA localized in one or more resistant cells and providing additional treatment to the resistant cells based on the image of the ^{99m}Tc-GLA localized in the cells. The method may be used to detect and develop treatments for cells and tissue that are resistant to prolonged ischemic stress or cells that are resistant to one or more chemotherapeutic agents such as cells are part of a drug resistant tumor.

### CROSS REFERENCE TO RELATED APPLICATIONS

This application claims the benefit and priority to U.S. Provisional Application Ser. No. 60/516,081, titled Breast Tumor Imaging with TC-99m Glucarate, Attorney Docket 2863.1002-000, filed Oct. 31, 2003, the contents of which are incorporated herein by reference in their entirety.

### GOVERNMENT INTERESTS

The United States Government may have certain rights to this invention pursuant to work funded under grants from the NIH under Grant No. P41 EB002035 and Grant No. R24 CA83148.

### BACKGROUND AND SUMMARY

^{99m}Tc radiopharmaceuticals have been used for various applications in nuclear medicine due to the optimal ^{99m}Tc emission energy of 140 keV, its diagnostically and patient tolerant half-life of about 6 hours, and its availability from ⁹⁹Mo—^{99m}Tc generator systems. Scintimammography (SMM) is an adjunct to conventional mammography in identifying patients with breast cancer using several radiopharmaceuticals either currently available or still under investigation. For example, ^{99m}Tc-sestamibi (MIBI) scintimammography (SMM) has been shown to have clinical utility in identifying breast tumors and has been approved by the U.S. Food and Drug Administration as an adjunct to mammography in the detection of breast cancer. Compared to conventional mammography, ^{99m}Tc-sestamibi imaging has utility in patients with large amounts of glandular tissue and dense breasts.

Surgical procedures, which can be used to determine the state of a tissue, for example a tumor, are not desirable because they are expensive, involve trauma, and require healing by patients who are often already in a weakened or immune compromised condition. Additionally, because the phenotype of explanted cells are subject to the different selective pressures of tissue culture, and are no longer subject to the in vivo selective pressures in the patient, they may be subject to genotypic and phenotypic alteration that could confound diagnosis and treatment.

^{99m}Tc-sestamibi is a lipophilic monocationic radiopharmaceutical, which accumulates predominantly within the mitochondria and cytoplasm of cells on the basis electrical potentials generated across membrane bilayers. Overall institutional sensitivity and specificity in ^{99m}Tc-sestamibi imaging are 75.4% and 82.7% for breast cancer detection. The positive predictive value is 74.5% and the negative predictive value is 83.4%. Perhaps because of either poor gamma camera resolution or lower ^{99m}Tc-sestamibi uptake in the tumors, the sensitivity of ^{99m}Tc-sestamibi SMM for small lesions is not as good as for larger lesions. For example, the sensitivity for tumors under 1 cm in size was only 48.2%. The larger lesions may also be undetected by ^{99m}Tc-sestamibi SMM due to low cellular proliferation or overexpression of a multidrug resistance (MDR) gene, MDR1, which encodes for a membrane glycoprotein, P-glycoprotein (Pgp). Pgp acts as an energy-dependent drug-efflux pump and allows transport of a wide range of structurally and functionally unrelated cytotoxic drugs out of tumor cells. In addition to the drugs, many surrogate markers of Pgp function in vivo such as ^{99m}Tc-sestamibi can also be pumped out. Faster clearance of ^{99m}Tc-sestamibi has been observed in tumors that express Pgp compared with tumors that did not express Pgp. To facilitate the detection of tumors which transport drugs, radiotracer labels and radiopharmaceuticals, or other agents out of tumor cells, it would be desirable to have a radiopharmaceutical that is not readily pumped out of the tumor cells and that could be used to identify the location of these resistant tumors.

D-glucaric acid is a six-carbon dicarboxylic acid sugar and a natural catabolite of glucuronic acid metabolism in mammals. All mammals excrete glucaric acid (glucarate) as a physiological end-product. Glucaric acid can be readily radiolabeled with sodium pertechnetate, resulting in ^{99m}Tc-glucarate (GLA). ^{99m}Tc-glucarate (GLA) was originally designed for detecting early necrosis of the heart and brain. It was reported that ^{99m}Tc-glucarate could also concentrate in malignant breast tumors. Biodistribution studies indicated that primary breast tumors in patients were visualized at early imaging time with ^{99m}Tc-glucarate. In SCID mice with xenografted BT20 tumors, ^{99m}Tc-glucarate was found to have significantly higher uptake than ^{99m}Tc-sestamibi. Still, the mechanisms of ^{99m}Tc-glucarate uptake and accumulation in the breast tumors are as yet unknown.

Ischemia-reperfusion injury can occur in various tissues, for example myocardial ischemia-reperfusion injury can occur after various procedures including angioplasty or thrombolysis and can be more severe than transient myocardial stunning. It may be possible to modulate or mitigate the effects of myocardial ischemia-reperfusion injury using ischemic preconditioning. By using various drugs, chemicals, or myocardial ischemic preconditioning ((IPC) is a process by which exposure of myocardium to a short period of non-damaging ischemic stress), the myocardium may become resistant to the deleterious effects of subsequent prolonged ischemic stress from procedures like angioplasty or thrombolysis.

Experimental studies have shown the favorable targeting potential of ^{99m}Tc-GLA of severe ischemia or early necrosis of the heart and brain. In acutely injured cells, it is believed that ^{99m}Tc-GLA is associated with disruption of the myocyte and nuclear membranes, allowing free intracellular diffusion and electrochemical binding of the negatively charged GLA complex to positively charged histones. Without wishing to be bound by theory, this intracellular distribution is believed to be driven by the avidity of GLA to the nuclear protein and cytoplasmic proteins with a positive charge. In rat models with isoproterenol-induced myocardial infarction, ^{99m}Tc-GLA uptake in the infarcted hearts was found 6 times more than in normal rats. ^{99m}Tc-GLA has been proposed as a specific oncotic marker in the very early stages of myocyte injury but not as an apoptotic marker. It would be desirable to provide a radiopharmaceutical that could be used to determine the resistance or extent (amount, degree, area, density of stressed cells/tissue) of resistance achieved from an ischemic preconditioning pharmaceutical compound or procedure in a patient prior to subjecting the patient to a prolonged ischemic stress from procedures like angioplasty or thrombolysis.

To date, few studies have been done regarding ^{99m}Tc-GLA imaging in ischemic-reperfused myocardium with varied severity of injury and none have established its use in determining the extent of ischemic-reperfused injury. It would be desirable to provide a radiolabel and method for using it that could be used to determine the amount or severity of myocardial damage in the hearts with ischemia-reperfusion injury.

Versions of the present invention relate to imaging cells and collections of cells such as tissues, organs, or tumors with an imaging agent that is a radiopharmaceutical having a glucarate ligand where the agent localizes within the resistant cells, tissues, organs, or tumors. The characterization of the cells and tissues, for example the size, area, and shape, where the imaging agent or radiopharmaceutical is localized can be used to determine the extent of the resistance of the cells, tissue, or tumor. This information may be used to determine if a prior treatment has been effective or if a further or new treatment of the imaged area is warranted in a patient and may also be used for the development of pharmaceutical compositions. Preferably the imaging agent is localized in a cells or a collection of cells the that can be characterized as being resistant cells. For example, in one version of the invention the cells or tissue that the imaging agent localizes are resistant to the deleterious effects of prolonged ischemic stress. In another version the cells that the imaging agent localizes are resistant to one on or more cytotoxic agents. The extent of the resistance, area or volume of resistant cells or tissue or severity can be determined by measuring the extent of the location of the ^{99m}Tc-GLA in the tissue or cells. Correlating the image of the agent accumulated in the tissue with the severity or resistance of the cells or tissue can be used to modify or develop a pharmaceutical compositions to treat the tissue. Alternatively, the image can be used decide if sufficient treatment was achieved in the patient and may be followed by the act of further treating the patient.

One version of the present invention is a method of characterizing a resistant tissue that includes imaging ^{99m}Tc-GLA localized in one or more resistant cells, tissue, or tumor and providing additional treatment to the resistant cells, tissue, or tumor based on the image of the ^{99m}Tc-GLA localized in the cells, tumor, or tissue. The size, area, and intensity of the image may be used to determine the amount of resistant cells, tumor, or tissue present in the patient. The size, area, or intensity of the image may be used to determine the severity or extent of an injury to cells, organ or tissue present in the patient. The tissue or cells may be those which are resistant to prolonged ischemic stress, or they may be resistant to one or more chemotherapeutic agents. The method and imaging agent may be used where one or more resistant cells are part of a drug resistant tumor. The method and imaging agent may be used where the one or more resistant cells are ischemic preconditioned myocardial cells. Preferably the imaging agent when used to detect drug-resistant cells or tumors, has nearly the same imaging kinetics in both drug sensitive and drug resistant tumors or cells.

Another version is of the invention is a method for detecting a treatment resistant tumor, and preferably a drug resistant tumor that includes but is not limited to the acts of: detecting ^{99m}Tc-GLA localized in a treatment resistant tumor in a patient. The method may further include the act of treating the resistant tumor following detection and or determining the extent of the ^{99m}Tc-GLA containing agent localized in the resistant tumor. The resistant tumor can include those tumors that such as but not limited to actual tumors in a patient or xenografts of tumor such as a drug resistant breast cancer tumor, a drug resistant lung cancer tumor, or a drug resistant myeloma. Preferably the method for detecting the resistant tumor in a patient, includes but not limited to mammals and humans, and can be used to detect and or determine the extent of a drug resistant breast cancer tumor. In the process of treating the patient, the method may also include the act of determining Pgp expression in the patient. The detection of Pgp expression in the patient may be used as an indication that the patient is at risk for having a treatment resistant tumor which may be imaged and evaluated with the ^{99m}Tc-GLA containing agent. The detection of the treatment resistant tumor with the imaging agent may follow or be part of a comprehensive course of treatment of the patient to eliminate the resistant tumor from the patient. The present invention can be used to identify various types of cancers in mammalian patients as well as to distinguish treatable tumors from treatment resistant tumors.

Another version of the invention is a method that includes the acts of correlating the severity of an ischemic-reperfusion injury in a tissue, preferably the severity of a myocardial injury in a patient, with the amount of ^{99m}Tc GLA localized in the tissue or myocardium of the patient. The injury may be a myocardial injury is caused by ischemic preconditioning, pharmaceutical preconditioning, or a combination of these. The severity of the injury can be determined by measuring the extent, size, or emission activity of the location of the ^{99m}Tc-GLA in the tissue or myocardium. In a preferred embodiment of the method, the myocardial injury is induced by pharmaceutical preconditioning. Correlating the image of the agent accumulated in the myocardial tissue with the severity of an ischemic reperfused myocardium can be used to decide if sufficient preconditioning was achieved in the patient and may be followed by the act of catheterizing an artery of the myocardium. ^{99m}Tc-GLA hot spot imaging, which includes determining the size or radioactivity of the localized imaging agent, can be used for noninvasively determining infarct size. The method may further include comparing the preconditioned tissue imaged with ^{99m}Tc-GLA to normal tissue imaged with ^{99m}Tc-GLA to characterize the extent of cardioprotection provided to the patient. The method may further include monitoring the tissue after a predetermined length of time following administration of the imaging agent where the slower washout of the imaging agent correlates with the extent of injury and a higher radioactive retention.

Another version of the invention is a method of characterizing a patient comprising the acts of identifying a patient at risk of having cells that transport cytotoxic drugs out of the cells and administering to the patient a detectable amount of a ^{99m}Tc-GLA imaging agent that can localize in the cytotoxic drug transporting cells for greater than 3 minutes. The imaging agent can then be detected and used to determine the location and accumulation of these cells in the patient.

In another embodiment of the present invention, drug resistant tumors may be distinguished from drug-sensitive tumors in a mammalian patient by administering to the patient suffering from a tumor ^{99m}Tc-sestamibi and then detecting a time series of signals from ^{99m}Tc-sestamibi and processing the detected signals into a first series of tomographic images. The patient may then be administered an amount of ^{99m}Tc-glucarate which is detected in a time series of signals from ^{99m}Tc-glucarate that can be processed into a second series of tomographic images. The first and the second series of tomographic images can be compared and the difference in the detected signals from the images used indicate that the tumor is drug-resistant. In an alternative method a patient suspected or at risk for having a resistant tumor may be administered in a stepwise manner or with a mixture of imaging agents that can include a radiolabel that does not accumulate in resistant tumors and a radiolabel having a glucarate ligand that accumulates in the tissue and monitoring the decay of signal from the tissue.

Advantageously, and unlike other scintigraphic agents and methods for characterizing resistant tumors which involve administration of a reversing agent, imaging the sample, and then comparing the sample image to an image obtained when no reverse agent is present, the method and imaging agent of the present invention results in imaged tissues or organs which retain the imaging agent even in the presence of Pgp or other transport system proteins. Unlike imaging agents which are transported out of cells, the methods of the present invention permit the imaging of resistant and treatment resistant cells, tissues, and tumors.

### FIGURES

The file of this patent contains at least one drawing/photograph executed in color. Copies of this patent with color drawing(s)/photograph(s) will be provided by the Office upon request and payment of the necessary fee.

FIG. 1 ^{99m}Tc-glucarate FASTSPECT coronal images (right panel (B)) in a mouse with MCF7/S tumor xenograft on right thigh (left panel (A)) 20 minutes after injection. Tumor (arrow) was localized clearly with low radioactive background on ^{99m}Tc-glucarate images.

FIG. 2. (A) and (B): ^{99m}Tc-glucarate dynamic FASTSPECT images (serial transaxial slices) from a mouse with MCF7/S breast tumor (arrow). The tumor (arrow) was visualized within 5-min and stayed well-defined for at least 120 min after ^{99m}Tc-glucarate injection. The time after injection is shown in the lower right corner of each image; (C) and (D): ^{99m}Tc-glucarate dynamic FASTSPECT images (serial transaxial slices) from a mouse with MCF7/D40 breast tumor (arrow). The tumor (arrow) was well visualized during the period for dynamic acquisition as seen in the MCF7/S tumor above.

FIG. 3 ^{99m}Tc-glucarate FASTSPECT images 120 minutes after injection in a mouse with MCF7/D40 tumor xenograft on right thigh (A). Tumor (arrow) was well localized in all directions of tomographic images (B: coronal; C: sagittal; D: transaxial slices).

FIGS. 4(A) and (B): Dynamic FASTSPECT images from a mouse with MCF7/S breast tumor on right thigh (arrow) using ^{99m}Tc-sestamibi (serial sagittal slices). The tumor was unequivocally visualized about 10-min and remained detectable until 120 minutes after injection; (C) and (D): ^{99m}Tc-sestamibi dynamic FASTSPECT images from a mouse with MCF7/D40 tumor xenograft on right thigh (serial sagittal slices), the tumor was visualized only 2-4 minutes after injection. The tumor radioactivity dropped quickly to the background level.

FIG. 5 Time-activity curves of ^{99m}Tc-sestamibi and ^{99m}Tc-glucarate in the MCF7/S and MCF7/D40 tumors. The radioactivity in the tumor with ^{99m}Tc-glucarate was significantly higher than that with ^{99m}Tc-sestamibi. In contrast to ^{99m}Tc-sestamibi, the radioactivity from ^{99m}Tc-glucarate was not considerably lower in MCF7/D40 that in MCF7/S tumors; MIBI: ^{99m}Tc-sestamibi; GLA: ^{99m}Tc-glucarate; S: MCF7/S; D40: MCF7/D40; *P<0.05 compared to MIBI/S. Error bar=SEM.

FIG. 6 The peak uptake and end radioactivity detected by FASTSPECT imaging using ^{99m}Tc-glucarate were significantly higher than that using ^{99m}Tc-sestamibi in both of the MCF7/S and MCF7/D40 tumors. The end activity of ^{99m}Tc-sestamibi in the MCF7/D40 tumors was significantly lower than that in the MCF7/S tumors. MIBI: ^{99m}Tc-sestamibi; GLA: ^{99m}Tc-glucarate; S: MCF7/S; D40: MCF7/D40; *P<0.05 compared to MIBI/S.

FIG. 7 ^{99m}Tc-glucarate and ^{99m}Tc-sestamibi washout curves from the MCF7/S and MCF7/D40 breast tumors. The radioactivity is plotted as a percentage of the peak activity in the tumor and extended to two hours using the TableCurve curve-fitting calculation. MIBI: ^{99m}Tc-sestamibi; GLA: ^{99m}Tc-glucarate; S: MCF7/S; D40: MCF7/D40; P<0.05 compared to MIBI/S.

FIG. 8 Illustrate experimental protocols in Example 2 for group 1(A) and group 2(B).

FIG. 9 Shows dynamic tomographic images (short axis) of ^{99m}Tc-GLA in heart sunjected to 30 min ischemia followed by 30 min reperfusion from 1 to 120 after injection. Blood pool was shown in 1 min image, an unequivocal hot spot was found on anterior and laterial wall of left ventricle from 10 to 120 min.

FIG. 10(A) ^{99m}Tc-GLA kinetic washout curces from normal and ischemic-reperfused areas in group 1 (IR30) and group 2 (IR90) hearts in Example 2. Curves were normalized to 100% of initial peak activity. *P<0.05 compared with IR90. (B) Ratios of hot spots wo viable myocardial ^{99m}Tc-GLA activities over time from hearts of group 1 (IR30) and group 2 (IR90). *P<0.05 compared with IR90.

FIG. 11 Hot spots were exhibited on ^{99m}Tc-GLA images (bottom row) from representative IR 30 heart (A) and IR90 heart (B) subjected to 30-min and 90-min ischemia treatments followed by reperfusion, which was consistent with myocardial IARs evaluated by Evans blue (unstained by blue dye) (top row) and infarcts determined by TTC staining (unstained) (middle row). Size of infarct and hot spot was relatively smaller in IR30 heart that that in IR90 heart.

FIG. 12 Scatter plot illustrates relationship between infarct measurements by ^{99m}Tc-GLA FASTSPEC imaging and TTC staining.

FIG. 13 Normalized ^{99m}Tc-glucarate time-activity curves in infarcted area (hot spot).

FIG. 14 Measurements of myocardial ischemia area at risk (IAR) and Infarct IAR (ischemic area at risk): unstained by Evans blue dye Infarct size: unstained by TTC

FIG. 15 Illustrates experimental results of adenosine mediated cardioprotection in rat hearts with ischemia-reperfusion: Adenosine A1 receptor activation by CCPA (left) Adenosine A1 receptor blockade by SPT (right); Adenosine receptor-mediated cardioprotection in rat hearts with ischemia-reperfusion: (A) Myocardial ischemic area by Evans Blue; (B) Myocardial infarct by TTC staining; (C) ^{99m}Tc-glucarate hot spot by FASTSPECT imaging.

### DETAILED DESCRIPTION

Before the present compositions and methods are described, it is to be understood that this invention is not limited to the particular molecules, compositions, methodologies or protocols described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention.

“Optional” or “optionally” means that the subsequently described event or circumstance may or may not occur, and that the description includes instances where the event occurs and instances where it does not.

Imaging agents, radiopharmaceuticals, or radiolabels used in versions of the present invention include those that have a radionuclide and one or more glucarate ligands and which localizes within resistant cells, tissues, organs, or tumors. Examples of useful tumor imaging agents include technetium labeled glucarate as disclosed in U.S. Pat. No. 4,946,668 and U.S. Pat. No. 4,952,393, the contents of which are incorporated by reference in their entirety into the present disclosure. The imaging complexes used in the present invention are derived from glucarate containing radionuclide complex compounds characterized in having reduced transport from the target cells or are retained by the target cells, tumor, or tissue. The imaging agents may be derived from mammalian physiological end-products and a preferred imaging agent is derived from D-glucaric acid. Preferably the imaging agent does not bind to non-target sites and clears faster from the circulation of the patient so that there is not so much background during imaging. The imaging agent can be characterized as having a biphasic washout from normal tissue such as from a normal breast tissue or normal myocardial tissue and increased retention, as measured for example by peak emission activity, in resistant tissue such a drug resistant tumor or ischemically-reperfussed preconditioned tissue. The imaging agent is not a substrate for MDR, and MDR is not a factor affecting the accumulation of the imaging agent in resistant tumors, cells, or tissues.

Unlike other scintigraphic agents which must be administered with a reversing agent, imaged, and then compared to an image obtained when no reverse agent is present, the method of the present invention results in imaged cells, an aggregate of cells usually of a particular kind together with their intercellular substance that form tissues, organs, lesions, or tumors that retain the imaging agent even in the presence of Pgp or other transporter system proteins. Unlike imaging agents which are transported out of cells, the methods of the present invention permit the imaging of resistant and treatment resistant cells, tissues, and tumors.

The method of the present invention can include the co-administration of the imaging agent with other pharmaceuticals or labels which may include but are not limited to chemotherapeutic agents, pharmaceuticals for ischemic-reperfusion preconditioning, or other radiolabels.

Prior to imaging, there may be indications or clinical signs of that a tissue may be resistant or that a patient is at risk of having a resistant tissue. For example a patient, which may be a mammal including mice or humans, may have had undergone a chemical pharmaceutical ischemic reperfusion preconditioning treatment or had an organ transplant. The patient or tissue may be examined or tested for the presence of a marker, like Pgp, suggesting drug treatment resistance or may have shown no improvement in health with a treatment regimen. In such cases an imaging agent that localizes to the resistant cells or tissue, preferably a glucarate containing technetium complex, can be administered and an image of the cells or tissue taken. Detection of the imaging agent localized to the site of the resistant cells or tissue may be characterized by hot spot imaging to determine the severity and extent of the resistant cells or tissue. The imaging agent localized to the resistant cells, tissue, or organ may be quantified using a region of interest established over the hot spot region where the glucarate radiopharmaceutical is located; quantification of the image may include background and decay correcting the images.

After the act of identifying the extent of the resistant sample of cells, tissue, or organ, the sample or patient may be subject to further treatment such as but not limited to surgery, radiation therapy, use of new drug or drugs with chemosensitizers or reversing agents that causes the accumulation of the chemotherapeutic, use of prolonged machine support, an angioplastic or mechanical thrombolysis, the implant of a stent, or a surgical procedure including a biopsy. Additional imaging administrations of the treated cells or tissue may be performed to determine whether the new treatment has improved the condition of the patient or whether the new treatment can be stopped.

The glucarate containing radiopharmaceutical imaging agent, preferably ^{99m}Tc-GLA, can be used to identify compounds and procedures for treating drug resistant tumors. Model animal studies can be performed where the animals are provided with xenografted drug resistant tumors. The tumor may be characterized by administering the imaging agent to the animal and measuring the extent of the localization of the imaging agent in the tumor. Various drug resistant tumor treating compositions or pharamaceuticals can then be administered to the animal and a determination of the extent of tumor reduction causde by the compositions based upon the amount or extent of localization of the imaging agent to the tumor tissue. The pharmaceutical compositions may vary in concentration, components of the composition (including reversing agents), or structure of the molecules that make up the composition. A reduction in the amount of imaging agent localized in the tumor tissue indicates that the test compound provided therapeutic benefit to the tissue. The action of test compounds in animal models can be correlated with the extent of localization of the imaging agent in the model drug resistant tumor and can be used to develop new compounds or select compounds for manufacture and or for use in patients.

A method for developing compounds and pharmaceutical compositions for treating drug resistant tumors can include the acts of providing a patient with a pharmaceutical composition for treating drug resistant tumor in the patient. A change in the size of the drug resistant tumor, based on an amount of ^{99m}Tc GLA localized in the drug resistant tumor tissue of the patient, can be correlated with the activity of the pharmaceutical. For example, a drug resistant tumor whose initial size has been characterized by ^{99m}Tc GLA localization and hot spot imaging may be treated with an administered pharmaceutical composition over a period of time to reduce the size of the drug resistant tumor. The reduction in the size of the tumor over the treatment period can be determined by measuring the extent of the hot spot from of the ^{99m}Tc-GLA localized in the tissue. The ability of the pharmaceutical composition to reduce the drug resistant tumor size can be modified by changing the pharmaceutical composition to increase or decrease its action against the drug resistant tumor. A patient having a drug resistant tumor can be treated with a pharmaceutical composition characterized by this method.

One method for detecting the multidrug resistance tumor in tissues in vivo is by detecting a drug resistant marker protein, such as Pgp, in a patient, and then administering a radiopharmaceutical imaging agent having a glucarate ligand that is retained by the resistant tissue and has washout kinetics for the imaging agent similar to those for a tumor that is treatable or is not drug resistant. Images of a drug resistant tumor can be used to evaluate the effectiveness of a course of treatment, and direct modification of the treatment, for tumors which have become multidrug resistant during the treatment. The imaging agents and methods of the present invention may also be used in vitro to study cytotoxicity in screening protocols for new cytotoxic compounds or in tissue biopsies from cancer patients to determine the amount and identity of effective cytotoxic agents for particular drug resistant tumor types.

Once a tissue or cells have been identified as being resistant and their location determined, a chemosensitizer or reversing agent may be administered as part of a therapeutic treatment. These agents are compounds that allows the net accumulation of toxic compounds in multidrug-resistant cells. Typical reversing agents include verapamil and quinidine. However, the invention can be practiced with any agent that reverses the multidrug resistance phenotype. Examples of alternative reversing agents include, but are not limited to vinblastine, vincristine, adriamycin, colchicine, daunomycin, dactinomycin, vanadate, cyclosporine and tetraphenylborate.

Myocardial ischemic preconditioning (IPC) is a process by which exposure of myocardium to a short period of non-damaging ischemic stress leads to myocardial resistance to the deleterious effects of subsequent prolonged ischemic stress. Myocardial Ischemia-Reperfusion Injury can occurs after angioplasty or thrombolysis. Mild ischemia can be used to protect the myocardium from effects of later ischemia and the protection appears biphasic: early (2-3 hours) and late (up to 3 days). Protection may be provided to tissues using physical treatment or chemical treatment of the tissue. The extent of protection provided to a tissue or sample may be determined by administering a glucarate containing radiopharmaceutical imaging agent, preferably ^{99m}Tc-GLA, and imaging the tissue to determine the extent of the localization of the imaging agent in the preconditioned tissue. The extent of cardioprotection provided to a patient to minimize or reduce ischemic-reperfusion injury to the myocardium can be determined thorough imaging of an administered glucarate containing radiopharmaceutical imaging agent that is localized in the preconditioned myocardial tissue. Protection may be provided to a tissue, and preferably cardioprotection, by physical ischemic preconditioning or chemical/pharmaceutical preconditioning including but not limited to administering adenosine, diazoxide, nitric oxide donor compounds or combinations of these to the patient or tissue.

Scintigraphy refers to the production of two dimensional or three dimensional reconstructed images of the distribution of radioactivity in tissues after the internal administration of an imaging agent, a radionuclide, or a radiopharmaceutical, the images being obtained by one or more scintillation cameras or detectors. The cameras may be rotated about the sample or patient where an image is reconstructed from the gamma rays or beta particles emitted from the agent in the sample. The cameras or detectors may be distributed about the patient or sample and an image reconstructed from the individual cameras or detectors. Preferably, the imaging system is a dedicated imaging system with stationary camera modules and a stationary multiple-pinhole aperture. For tomographic imaging with fixed cameras, no center-of-rotation corrections are required, and artifacts caused by inaccurate center of rotation are not present. The imaging system preferably provides fast sequential images combined high sensitivity and high spatial resolution. High-resolution imaging using pinhole or multiple-pinhole gamma camera system are available for clinical use and small-animal studies and can be used to investigate lesions of less than 1 cm. The imaging system and imaging agent provide an approach for tumor targeting, quantitative 4-dimensional imaging, and radiopharmaceutical in vivo kinetic studies using small size xenografted tumors with less necrosis. In alternative methods of the present invention, agents are not detected by imaging cameras but by quantitative measurement of the intracellular accumulation (e.g., in a gamma counter or as by scanning radiograms by densitometry). Whole body imaging in patients with tumors has previously demonstrated uptake of technetium radiolabels within mediastinal and pulmonary metastasis from thyroid cancer, untreated malignant lung lesions and known bronchial carcinomas.

The radiopharmaceutical imaging agent having a glucarate containing nuclide can be injected into a vein and absorbed by healthy tissue at a known rate during a certain time period. The radionuclide detector, for example a gamma scintillation camera, picks up the gamma rays emitted by the isotope. After the imaging agent, for example technetium glucarate, is injected into a blood vessel in the arm, it accumulates in resistant tissue such as a drug resistant tumor or heart tissue that has been damaged. The accumulated imaging agent in the resistant tissue a leaving “hot spots” that can be detected by the scintillation camera. The patient can be placed on test table that is rotated during imaging so that different views of the tissue with the localized imaging agent can be scanned. The camera, which looks like an x-ray machine can be suspended above the table and move back and forth over the patient. The imaging system displays a series of images of technetium's localization in the patient tissue and records them on a computer for later analysis.

In embodiments of the present invention, the radiolabel having one or more glucarate ligands and that can become localized in resistant cells, tissues, or tumor is preferably imaged by defining a region of interest (ROI). The ROI can be established over a hot spot, a portion of the sample emitting gamma rays or high energy beta particles (a signal) from the radiolabel, with the highest activity. For example, in the case of ^{99m}Tc-GLA radiolabel localized to a sample of cells, a tissue, or a tumor, the highest radiolabel activity (counts/pixel) can be selected from a short-axis slice of the 120-min, or some other convenient and useful time interval image. The ROI can be applied to all images in the time interval, such as from 1 to 120 min after percutaneous administration of the radiolabel to the sample or patient. After correction for background and emission decay, ^{99m}Tc gamma ray decay, the radioactivity (counts/pixel/min) in the hot spot can be projected onto the dynamic images to determine the time-activity curve for the radiopharmaceutical having one or more glucarate ligands that is localized in the sample. Using an ROI selected from a normal cell or normal tissue sample having the radiolabel present, a normal sample or control time-activity curve can be generated and compared with the time activity curve for the radiolabel in the resistant cells, tissue, or tumor. Hot spots on the short-axis slices of 120-min image can be quantified with ROI analysis to calculate the ratios of resistant cells or tissue zone activities to normal cell sample or normal tissue sample activities and averaged over the whole sample to produce a mean ratio.

A new growth of tissue in which the multiplication of cells is uncontrolled, progressive, and resistant to treatment is referred to as a resistant tumor. Treatment resistant tumors that are particularly relevant in embodiments of the present invention are the drug resistant tumors that are associated with expression of markers including but not limited to MDR1, or Pgp. The detection of Pgp expression in a patient or tissue sample undergoing a course of chemotherapy may be used to indicate the presence of a drug or multidrug resistant tumor and a glucarate based radionuclide imaging agent used to identify the location and extent of the resistant tumor in the sample or patient.

Using the methods and materials of the present invention it is not only possible to distinguish between acute ischemic reperfusion tissue injury and normal tissue or older injuries, but it is possible to correlate the degree or severity of the ischemic reperfusion injury. Where the injury is caused during a course of ischemic reperfusion pre-conditioning, the degree of the preconditioning may be evaluated by characterizing the area, intensity, and location of the imaging agent in the tissue, preferably the myocardium. Rather than just being able to detect an injury or no injury, the present method can be used to determine if treatment is needed, machine support for a transplanted organ, or if the tissue is sufficiently preconditioned for a subsequent procedure like angioplasty or mechanical thrombolysis. The method includes but is not limited to the steps of administering percutaneously to a patient an effective amount of a radiopharmaceutical having a glucarate ligand, preferably ^{99m}Tc-GLA, and then scanning the patient using a high-speed gamma camera or similar instrument to characterize the extent of the localized ^{99m}Tc-GLA.

The glucarate containing radionuclide complex imaging agent, preferably ^{99m}Tc-GLA, can be used to identify compounds and procedures that provide ischemic preconditioning. Model animal studies can be performed where the animals are provided with various ischemic preconditioning compositions or pharmaceuticals and then determining the extent of ischemic preconditioning based upon the amount or extent of localization of the imaging agent to a tissue, preferably the myocardium, of the animal. The pharmaceutical compositions may vary in concentration, components of the composition, or structure of the molecules that make up the composition. A large localization of the imaging agent to the tissue indicates that the test compound provide ischemic preconditioning to the tissue. For example, the imaging agent and detection methodology can be used to determine whether adenosine A1 receptor is involved in protection induced by ischemic preconditioning in the rat heart model and used to screen or develop drugs for human use or trials. If the adenosine receptor is involved, an adenosine receptor like agonist, should give protection similar to ischemic preconditioning, while an A1 receptor antagonist, should block protection by ischemic preconditioning. The action of test compounds in animal models can be correlated with the extent of localization of the imaging agent in the model heart can be used to develop new compounds or select compounds for manufacture and use in patients.

A method for developing compounds and pharmaceutical compositions for ischemic preconditioning can include the acts of providing a patient with a pharmaceutical composition and correlating the severity of an ischemic reperfussion injury caused by the pharmaceutical composition in a tissue of the patient with the amount of ^{99m}Tc GLA localized in the injured tissue of the patient. For example, the ischemic reperfusion injury may be a myocardial injury related to myocardial preconditioning. The size of the injury can be determined by measuring the extent of the hot spot from of the ^{99m}Tc-GLA localized in the tissue. The extent of the preconditioning or reperfusion injury can be modified by changing the pharmaceutical composition to increase or decrease the ischemic reperfusion injury. A patient can be treated with a pharmaceutical composition for ischemic preconditioning that has been characterized by this method.

To image resistant cells, tissue, or tumors in mice, a volume of solution containing a radionuclide having one or more glucarate ligands, preferably ^{99m}Tc-GLA, sufficient to deliver between about 9 to 10 MBq of ^{99m}Tc-GLA/kg body weight can be injected intravenously into the mice. With humans, it is reasonable to expect that injection of a volume of ^{99m}Tc-GLA solution containing about 370-740 MBq into the patient to be imaged (5 to 10 MBq/kg body weight) will be suitable for imaging tissue in the patient. The imaging agent can be administered percutaneously, for example intravenously, and preferably no more than about 3 hours after preparation. The subject is then scanned. Preferably, scanning is conducted between 1 and 6 hours after radiolabel administration to the sample.

The methods of the present invention are also applicable to whole tissues, biopsy samples, and cells in vitro. The invention is advantageous over current methods of determining the multidrug resistance phenotype in vitro because it is rapid and simple. Using presently available methods, before a multidrug resistance phenotype can be evaluated in whole tissue, a single cell suspension must be created (e.g., for flow cytometry) or even more laborious techniques must be used, such as monolayer cell culture. Using the method of the current invention, it is possible to detect the multidrug resistance phenotype in tissue without, or with minimum, disaggregation. Thus, therapeutic regimens may be decided with less delay than with presently available methods.

Some in vitro procedures for detecting the multidrug resistance phenotype involve forming either cell monolayers or single cell suspensions because the detectable emission (i.e., beta rays or fluorescence) does not penetrate and pass through intervening biological material. Thus, there is no rapid procedure for assaying the multidrug resistance phenotype of cells in a tissue or a cell mass, such as a tumor or tumor biopsy. Tissue would have to be dispersed into single cells for analysis and may have to be cultured. Cell culture, however is time consuming and also alters the selection pressures so that the cultured cells do not display the same phenotype or genotype as the cells in vivo. For example, the overexpression of the multidrug resistance gene in a tumor occurs as a result of the selection and multiplication of single or a few mutant cells as the tumor is subjected to a chemotherapeutic drug. If the tumor is excised and grown in tissue culture, the genotype may change because the selection pressure is not the same. This may interfere with the proper analysis of the tumor and hence with prescription of a effective therapeutic regimen. With the methods of the present invention, however, the tumor could be analyzed without dispersion and growth in culture.

Tumors are usually genotypically and phenotypically heterogeneous. New genotypes may arise in a very small or minute portions of a tumor and may not be detectable by routine methods. For example, the multidrug resistance phenotype occurring in a small area of a tumor, may be missed if the tumor cells are dispersed or merely biopsied. With the methods of the present invention, since a small area would be intact, imaging the tumor would reveal such small pockets of multidrug resistant cells. One method for obtaining tomographic imaging of a resistant tumor in a mammalian patient comprises administering ^{99m}Tc-glucarate to a mammalian patient suffering from a tumor; detecting a signal from the localized ^{99m}Tc-glucarate; and processing the detected signal into a tomographic image using a region of interest and optionally background and decay correction. Drug-resistant tumors, suggested by the detection of an expressed marker or from cell culture resistance testing, of the breast, lung, or bone (myeloma) may be imaged by this method.

Another method of distinguishing drug-resistant tumors from drug-sensitive tumors in a mammalian patient can include administering ^{99m}Tc-sestamibi to the patient suffering from a tumor; detecting a time series of signals from ^{99m}Tc-sestamibi; processing the detected signals into a first series of tomographic images; administering ^{99m}Tc-glucarate to the patient suffering from the tumor; detecting a time series of signals from ^{99m}Tc-glucarate; processing the detected signal into a second series of tomographic images; and comparing the first and the second series of tomographic images. The difference in the detected signals of the later images of the series indicate that the tumor is drug-resistant.

Accordingly, the invention embodies methods of assaying the multidrug resistance phenotype in whole tissue or tissue biopsies by incubating the tissue or biopsy with the agents of the present invention. In alternative embodiments, the glucarate radiopharmaceutical imaging agent is administered alone and the measurement obtained is compared with the measurement obtained with normal control tissue.

The invention also embodies methods of designing treatment regimens for resistant cells, tissues, or tumors. For example, a patient in need of a transplant, angioplastic or thrombolytic procedure may be subject to ischemic-reperfusion preconditioning prior to the procedure. The method of the present invention may be used to determine if sufficient ischemic-reperfusion preconditioning was achieved in the patient or a transplant tissue based on the amount of signal obtained from localized glucarate containing radiolabel and quantitative analysis using ROI and hot spot imaging. In another example, the effect of chemotherapy regimens on resistant tumors, especially multidrug resistant tumors may be monitored by assaying the multidrug resistance phenotype in patients or their explanted tissue either prior to or during treatment. During the course of chemotherapy, when it is determined that a multidrug resistant tumor is present based on the detection of a marker and localization of a glucarate containing radiopharmaceutical in the tumor, a change in the tumor treatment can be made. The sample or a patient can be screened for the expressed Pgp and the location, size, and portion of the tumor that is drug resistant determined by a glucarate containing radiopharmaceutical, preferably ^{99m}Tc-GLA, localized in the drug resistant portion of the sample. The treatment for a patient can be made based on historical experience with different drug regimens or therapies, such as but not limited to radiation, surgery, mechanical support, angioplasty, and thrombolysis, and the patient's general medical condition including age, kidney function, and prior treatment history.

With the method of the present invention, it is also possible to evaluate in vivo the efficacy of alternative chemotherapeutic drugs. If the alternative chemotherapeutic drug is able to reverse the multidrug resistance of the tumor, the area to which the imaging agent is localized in the tumor will decrease as will the expression of Pgp or other resistant tumor markers.

The reagents for performing the radiopharmaceutical imaging can be assembled in kits for convenient performance of the method in the clinic. The method may include the preparation of the active radiopharmaceutical like ^{99m}Tc-glucarate (^{99m}Tc-GLA). The kit may include a identifiable vial for sampling a fluid of the patient for an expressed resistance marker, and reagents for radiolabeling a glucarate ligand with the a radionuclide that can consist of a sealed and sterile vial containing reducing agent (preferably stannous ions) and the carbohydrate ligand (preferably glucaric acid) in aqueous solution. The preferred radionuclide containing compound, a pertechnetate ion, is added to the vial containing the reducing agent and the ligand. The contents are then mixed and incubated for a time sufficient to effect labeling of the ligand. The radiolabeled ligand can then be used immediately without purification. An apparatus that may be used for detecting absorption of a labeled pharmaceutical by resistant cells can include one or more stationary or movable signal detectors; and a kit that includes at least one signal-producing labeled pharmaceutical such as ^{99m}Tc-glucarate or ^{99m}Tc-glucarate and ^{99m}Tc-sestamibi. The signal detector can be a tomographic imaging system.

Tomographic imaging of a resistant tissue, for example a drug resistant tumor or a ischemic preconditioned tissue, may include administering a signal-producing labeled pharmaceutical to the tissue, detecting the signal from the signal-producing labeled pharmaceutical and processing the detected signal to generate a tomographic image. In a preferred embodiment, the tissue is a tumor in a mammalian patient, the signal-producing labeled pharmaceutical is ^{99m}Tc-glucarate and an imaging system having one or more detectors, which can be stationary or movable, is used to detect and process the signal from the agent localized in the tissue. An advantageous process for tomographic imaging of a tumor in a mammalian patient can include administering ^{99m}Tc-glucarate to a mammalian patient suffering from a resistant tumor that may be indicated by detection of an expressed marker or by cell culture drug resistance testing, detecting a signal from ^{99m}Tc-glucarate localized in the patient, and processing the detected signal into a tomographic image. Drug-resistant tumors include those including but not limited to breast, lung, or a myeloma.

Detection of the radiopharmaceutical having a glucarate ligand can be achieved using detectors capable of detecting signals such as emitted gamma rays or beta particles, preferably high energy beta particles, emitted by the radionuclide localized in the resistant cells that can pass from the cell, tumor, or tissue sample to the detector.

In the MDR phenotype, several proteins have been determined to be over expressed in this phenotype and these proteins can be used as markers for the MDR phenotype. P-glycoprotein, Pgp, is a protein expressed by the MDR1 gene in humans. MDR1 is one of two multi drug resistance (MDR) genes in humans, while rodents have three. The MDR gene products are members of a “superfamily” of transporters known as ABC (ATP-binding cassette) proteins. ABC transporter proteins traverse membranes in order to transport molecules out of or within cells. These molecules are so highly conserved in evolution that some of them can substitute effectively for one another in species ranging from bacteria to yeast to mice to humans. MRP for multidrug resistance-associated protein, is—like Pgp—a member of the ABC superfamily and is located mainly in the plasma membrane of resistant cells and can act by removing drugs from cells. Close relatives of MRP have been found in yeast, nematode worms, rabbits, and humans.

A tumor which is sensitive to a given drug will have an above-average probability of being destroyed or damaged by the drug, and the patient will have an above-average probability of deriving benefit from treatment with the drug, all other factors being equal (where other factors includes whether or not the patient's medical condition is consistent with the patient being able to safely receive the drug in question). A tumor which is resistant to a given drug will have a below-average probability of being destroyed or damaged by the drug, and the patient will have a below-average probability of deriving clinical benefit; drug resistance may be determined by cell culture drug resistance testing or the detection of the expression one or more markers like MPR or Pgp in the patient or sample.

Ischemia-reperfusion injury can occur in various tissues, for example implanted tissues may suffer ischemia-reperfusion injury and myocardial ischemia-reperfusion injury can occur after various procedures including angioplasty or thrombolysis. It would be beneficial in the treatment of a patient to be able to provide a physician with an indication of the extent or degree of ischemia-reperfusion preconditioning that has occurred or the extent of injury that has occurred following a tissue or organ implant or an angioplastic procedure. Based on the extent of the ischemia-reperfusion injury detected by a glucarate containing radiopharmaceutical, such as ^{99m}Tc-GLA or a composition including ^{99m}Tc-GLA, localized in the resistant cells and imaged, further treatments or delay of a scheduled procedure may be indicated.

Infarct is an area of dying or dead tissue resulting from obstruction of the blood vessels normally supplying the part or tissue. During tissue or organ transplant, for example livers, damage to the tissue or transplanted organ can occur when blood flow to the tissue or organ is restored to the after transplant. This damage occurs when ischemic, or oxygen-deprived, tissue is re-introduced, or reperfused, to adequate blood flow. Reperfussion stress can cause death of the tissue and methods to mitigate or prevent the damage can be used, for example nitric oxide.

Treating a patient with ^{99m}Tc-glucarate would permit visualization of reperfussion injury and facilitate prompt action such as mechanical support for an injured organ. ^{99m}Tc-GLA accumulation in myocardial tissue can be used to determine the amount, extent, and location of myocardial damage in a heart with an ischemia reperfusion injury. The in vivo kinetic properties of ^{99m}Tc-GLA, its retention in terms of emission intensity and the size of the tissues including the ^{99m}Tc-GLA hot spot, may be used to assess the severity of myocardial ischemia-reperfusion injury.

Mammography is a diagnostic procedure where an X-ray picture of the breasts is used to detect tumors and cysts and to help differentiate benign (noncancerous) and malignant (cancerous) disease. Scintimammography as described in the present invention includes methods for characterizing the size and extent of glucarate containing radiopharmaceutical localized in drug resistant tumors.

In blood plasma, the dissolved compounds have an osmotic pressure. A small portion of the total osmotic pressure is due to the presence of large protein molecules; this is known as the colloidal osmotic pressure, or oncotic pressure. Because large plasma proteins can't easily cross through the capillary walls, their effect on the osmotic pressure of the capillary interiors will, to some extent, balance out the tendency for fluid to leak out of the capillaries. In conditions where plasma proteins are reduced, e.g. from being lost in the urine (proteinuria) or from malnutrition, the result of too low an oncotic pressure can be edema—excess fluid buildup in the tissues.

Various aspects of the present invention will be illustrated with reference to the following non-limiting examples.

### EXAMPLE 1

This example shows the detection of a resistant tumor and determination of the tumor extent using a glucarate containing radiopharmaceutical, ^{99m}Tc-labeled glucarate, localized in a resistant tumor using multiple fixed detectors.

Western blot analysis indicated that human MDR1 Pgp was well expressed in the xenografted MCF7/D40 tumors. A prominent band was observed in tumor cell membrane preparations with C219 antibody indicating Pgp expression in the MCF7/D40 tumor samples. No immunodetectable MDR1 Pgp was presented in the xenografted MCF7/S tumor samples.

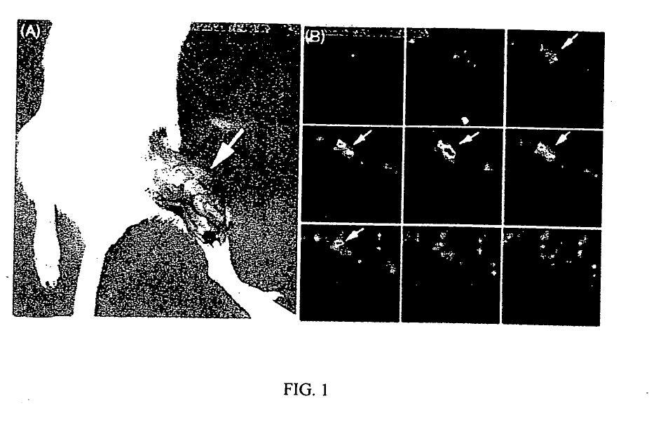

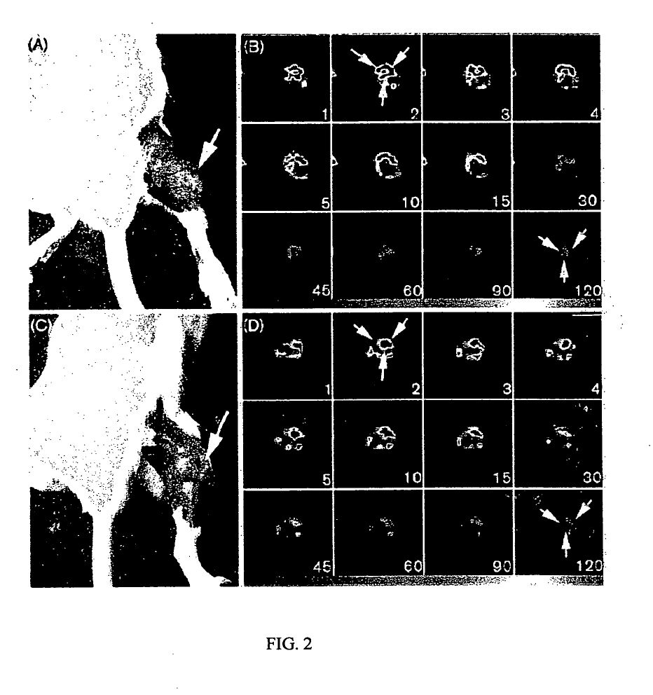

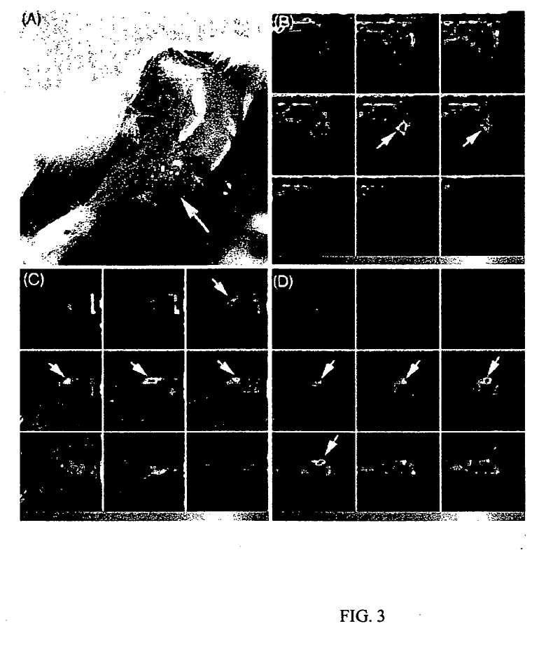

All tumors of ^{99m}Tc-glucarate groups were initially visualized within 5 min by FASTSPECT imaging after injection of ^{99m}Tc-glucarate, and unequivocally localized within 10-30 minutes. FIG. 1 shows representative ^{99m}Tc-glucarate coronal tomographic images in a mouse with subcutaneous MCF7/S tumor 20 minutes post injection. Dynamic ^{99m}Tc-glucarate images demonstrated that the radioactive accumulation in the MCF7 tumors remained for at least 120 minutes post-injection. Representative dynamic images in MCF7/S and MCF7/D40 are shown in FIG. 2. Relative to the drug-sensitive tumors, the drug-resistant tumors exhibited same level uptake of ^{99m}Tc-glucarate during the entire period for dynamic acquisition. Two-hour post administration, the MCF7/D40 tumor xenograft could be well visualized in all directions of tomographic ^{99m}Tc-glucarate images (see FIG. 3). In comparison with ^{99m}Tc-sestamibi imaging, ^{99m}Tc-glucarate imaging demonstrated lower background activity and higher tumor accumulation.

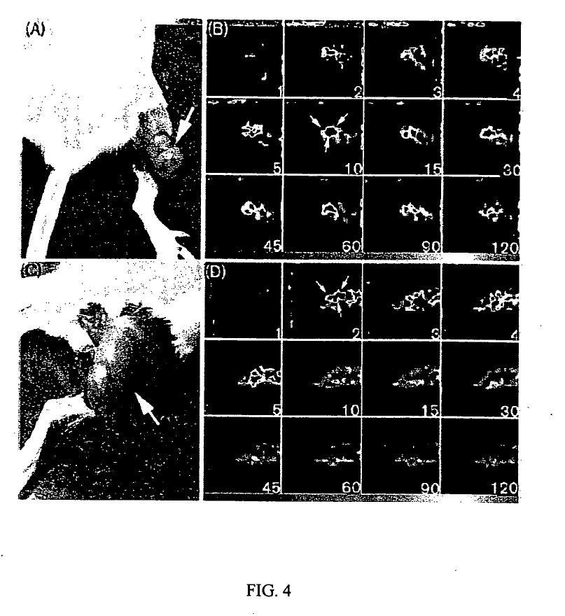

The MCF7/S tumors could be detected initially by FASTSPECT imaging 2-10 minutes after intravenous administration with ^{99m}Tc-sestamibi. Two-hour post injection, dynamic images demonstrated that tumor activity remained detectable (FIG. 4A and FIG. 4B). In contrast, as shown in FIG. 4C and FIG. 4D, the MCF7/D40 tumors could be localized only 2-4 min post injection of ^{99m}Tc-sestamibi; then the radioactivity in the tumors dropped quickly to background levels.

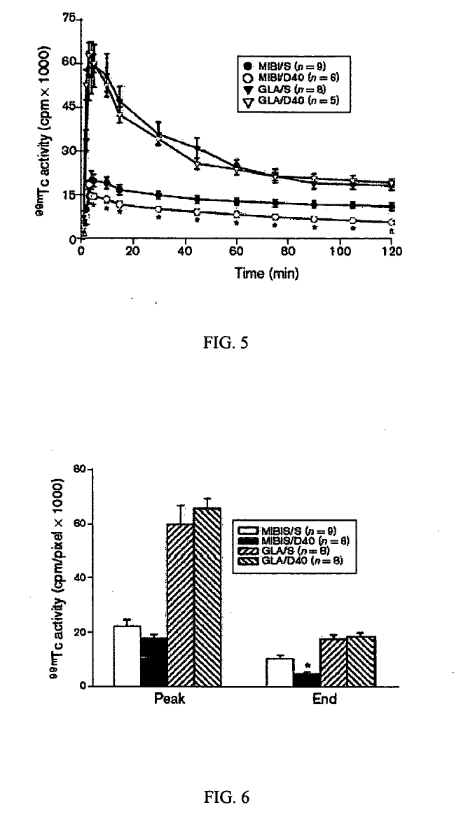

The kinetics of ^{99m}Tc-glucarate and ^{99m}Tc-sestamibi in MCF7 Tumors were determined. FIG. 5 shows time-activity curves plotted from 1 minute to 120 minutes for ^{99m}Tc-glucarate and ^{99m}Tc-sestamibi in the MCF7/S and MCF7/D40 xenografts. The activity of the imaging agents was determined by computerized ROI analysis of FASTSPECT images that were background subtracted and decay corrected. The time-activity curves of ^{99m}Tc-sestamibi were observed with a significant difference at each point in time from 5 minutes to 120 minutes between the MCF7/S and MCF7/D40 tumors. There was no difference at each point between ^{99m}Tc-glucarate time-activity curves of the MCF7/S and MCF7/D40 tumors. Significantly higher uptake in each xenografted tumor model was found for ^{99m}Tc-glucarate than for ^{99m}Tc-sestamibi (p<0.05); the peak uptake and end radioactivity (cpm/pixel×1000) of the tumors determined by FASTSPECT imaging and normalized by the injected dose were shown in FIG. 6.

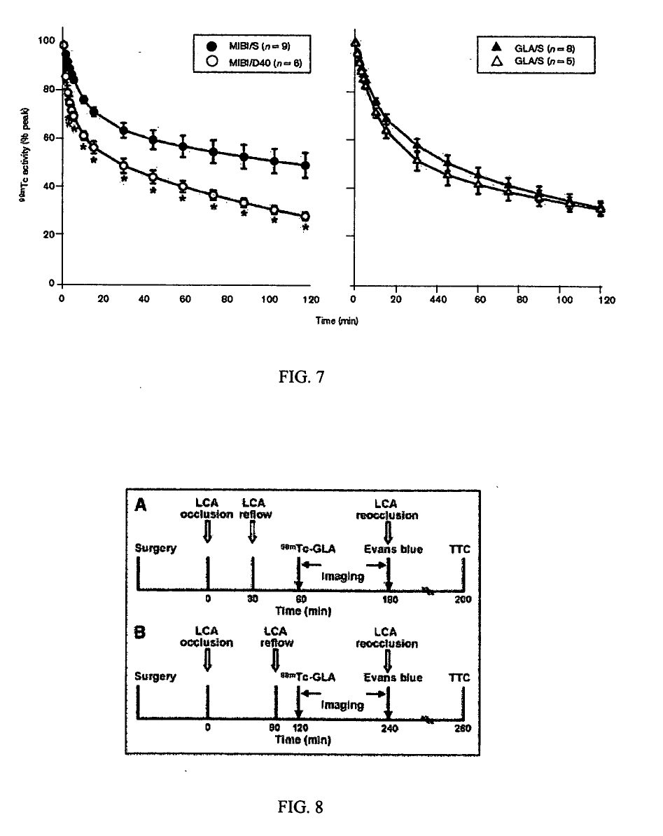

^{99m}Tc-glucarate and ^{99m}Tc-sestamibi fractional clearance curves are shown in FIG. 7; tumor activity is shown as a percentage of peak activity over two hours. The individual curves were fitted and extended to two hours using the TableCurve curve-fitting calculation. Both ^{99m}Tc-glucarate and ^{99m}Tc-sestamibi exhibited biphasic clearance curves, and biexponential equations were found to provide the best fits to the curves. The early phase showed fast clearance and the late phase showed slow clearance. The half-time values (minutes) of biexponential washout (t_{1/2}, time to reach half of initial activity) from the tumors are shown in Table 1.

TABLE 1
Biexponential Tumor Clearance Half-time (minutes)

| | a. ^{99m}Tc- | | b. ^{99m}Tc- | |
|---|---|---|---|---|
| | sestamibi | | glucarate | |
| | MCF7/S | MCF7/D40 | MCF7/S | MCF7/D40 |
| arly Phase | 11.4 ± 1.9 | 4.4 ± 1.4* | 12.2 ± 2.6 | 12.4 ± 2.3 |
| Late Phase | 431.3 ± 56.1 | 178.9 ± 21.5* | 170.1 ± 16.2 | 219.3 ± 38.8 |

mean ± s.e.m.,
*= p < 0.05 compared to MCF7/S.

There was a significant difference in the early phase t_{1/2} value for ^{99m}Tc-sestamibi between MCF7/S and MCF7/D40 (p<0.05). The late-phase t_{1/2} value for ^{99m}Tc-sestamibi in MCF7/D40 was significantly shorter than the value in MCF7/S. There were no differences in t_{1/2} values for the early and late phase of ^{99m}Tc-glucarate between MCF7/S and MCF7/D40. ^{99m}Tc-glucarate demonstrated a significantly shorter t_{1/2} in the late phase compared to ^{99m}Tc-sestamibi in MCF7/S. ^{99m}Tc-sestamibi in the MCF7/D40 tumors exhibited shorter t_{1/2} than ^{99m}Tc-glucarate in the early phase (p<0.05).

The two-hour fractional washout (% peak activity) of ^{99m}Tc-sestamibi was demonstrated significantly greater in MCF7/D40 than that in MCF7/S (71.7±1.7% vs. 50.2±5.3%, p<0.05), but there was no difference (71.9±2.9% vs. 72.8±3.1%, p>0.05) for ^{99m}Tc-glucarate.

TABLE 2
Biodistribution Data (% ID/gm)

| | a. ^{99m} | | b. ^{99m} | |
|---|---|---|---|---|
| | Tc-sestamibi | | Tc-glucarate | |
| | MCF7/S | MCF7/D40 | MCF7/S | MCF7/D40 |
| Blood | 0.06 ± 0.01 | 0.07 ± 0.01* | 1.13 ± 0.16^{†} | 1.07 ± 0.14^{†} |
| Muscle | 1.37 ± 0.12 | 1.09 ± 0.32 | 0.27 ± 0.02^{†} | 0.25 ± 0.04^{†} |
| Lung | 0.95 ± 0.16 | 0.61 ± 0.12 | 1.08 ± 0.10 | 1.41 ± 0.54 |
| Heart | 10.61 ± 0.89 | 9.78 ± 1.65 | 0.64 ± 0.08^{†} | 0.55 ± 0.10^{†} |
| Liver | 7.02 ± 1.01 | 7.67 ± 1.52 | 2.04 ± 0.37^{†} | 1.56 ± 0.18^{†} |
| Kidneys | 21.32 ± 2.03 | 17.71 ± 3.13 | 22.70 ± 1.38 | 22.10 ± 1.59 |
| Tumor | 0.64 ± 0.07 | 0.27 ± 0.05* | 1.15 ± 0.17^{†} | 1.39 ± 0.12^{†} |
| Tumor | 0.12 ± 0.05 | 0.11 ± 0.04 | 0.15 ± 0.03 | 0.10 ± 0.03 |
| weight | | | | |
| (gm) | | | | |

mean ± s.e.m..
*= p < 0.05 compared to MCF7/S;
^{†}= p < 0.05 compared to ^{99m}Tc-sestamibi.

The average tumor-weight for the 28 mice was 0.12±0.02 gram. No difference was found in tumor weight between the MCF7/S and MCF7/D40 tumors imaged with ^{99m}Tc-glucarate or ^{99m}Tc-sestamibi. The biodistribution data are shown in Table 2. ^{99m}Tc-sestamibi exhibited significantly lower radioactive accumulation (% ID/gm) in the MCF7/D40 tumors than that in the MCF7/S tumors, (p<0.05). ^{99m}Tc-glucarate showed significantly higher accumulation (% ID/gm) than ^{99m}Tc-sestamibi in either the MCF7/S or MCF7/D40 tumors. There was no difference in ^{99m}Tc-glucarate accumulations between the two kinds of xenografted tumor models. ^{99m}Tc-glucarate demonstrated significantly lower radioactivity in non-tumor soft tissue (muscle) compared to ^{99m}Tc-sestamibi (p<0.05). As a result, 2 hours after injection, the tumor-to-muscle ratios (tumor/muscle) for ^{99m}Tc-glucarate were 9.4-fold higher than that for ^{99m}Tc-sestamibi in the MCF7/S tumors, and 19.2-fold higher in the MCF7/D40 tumors. The blood activity of ^{99m}Tc-sestamibi was significantly lower than ^{99m}Tc-glucarate. Relative to ^{99m}Tc-sestamibi, ^{99m}Tc-glucarate showed a significant lower accumulation in liver.

The results show ^{99m}Tc-glucarate exhibited same kinetics in the drug-resistant tumors as in the MCF7/S tumors, which are sensitive to doxorubicin. Thus, MDR is not a factor affecting the accumulation of ^{99m}Tc-glucarate in the MCF7 breast tumors. The use of ^{99m}Tc-labeled glucarate for detecting MCF7 breast tumors was demonstrated and compared to ^{99m}Tc-sestamibi imaging using the high-resolution multiple fixed detector cameras. In the xenografted tumor models, ^{99m}Tc-glucarate showed higher tumor uptake than ^{99m}Tc-sestamibi. As described previously, ^{99m}Tc-sestamibi is a substrate for MDR in the drug-resistant MCF7 tumors. The expression of Pgp is responsible for the high washout rate of ^{99m}Tc-sestamibi.

^{99m}Tc-sestamibi is an isonitrile compound and is concentrated primarily in mitochondria. The uptake of cationic ^{99m}Tc-sestamibi depends on negative transmembrane and mitochondrial potentials. Although the lipophilic sestamibi might diffuse through plasma membranes by a passive mechanism, Na⁺/K⁺ ATPase pump activity maintains the membrane potential. It is fixed intracellularly as long as cell membrane integrity is intact and nutrient blood flow persists. Without wishing to be bound by theory, this active energy-dependent mechanism might explain ^{99m}Tc-sestamibi's slower and lesser washout from the tumor xenografts.

Since glucarate is a six-carbon dicarboxylic acid sugar and can behave as a glucose analogue, the increased uptake of GLA may relate to upregulated glucose transport in the tumor cells. Over expression of glucose transporters directly involves the accelerated metabolic processes to accommodate the energy requirements of rapidly dividing cells and provide the carbon backbone for DNA and RNA synthesis for cell proliferation in growing tumors. The uptake of ^{99m}Tc-glucarate in certain cells was inhibited by fructose. Insulin administration was found to increase ^{99m}Tc-glucarate uptakes in the rat hearts, livers and skeletal muscles significantly. These experimental results suggest that ^{99m}Tc-glucarate may enter certain cells by the sugar transport system.

The exact mechanism of ^{99m}Tc-glucarate uptake in tumor is currently unclear. In acute infarcted myocardium, ^{99m}Tc-glucarate was observed to target the nuclei of the necrotic myocardium. High nuclear uptake of ^{99m}Tc-glucarate was also reported in the BT20 human breast-tumor cell line; the mitochondrial and cytosolic fractions also sequestered a substantial amount of ^{99m}Tc-glucarate. However, the nuclear fraction of ^{99m}Tc-glucarate distribution in tumor cells was lower than that in infarcted myocytes. When [¹⁴C] glucarate was administrated to rats bearing primary mammary tumors, significant binding of glucarate to proteins was found in the cytosolic fraction. However, it is unclear how ^{99m}Tc-glucarate is transported into the nuclei and mitochondria. Experimental evidence suggests that the activity of glucarate is mediated via signal transduction pathways involving cAMP and protein kinase C. In acute infarcted myocardium, it is believed that ^{99m}Tc-glucarate is associated with disruption of the myocyte and nuclear membranes, allowing free intracellular diffusion and electrochemical binding of the negatively-charged glucarate complex to positively-charged histones. However, the uptake of ^{99m}Tc-glucarate associated with tumor necrosis or membrane breakdown is less likely in the current study, in which we used the xenografted tumors 10-14 days after tumor implantation. The tumor size was limited to about 0.12 grams. No significant tumor necrosis was found in histological and electron microscopic examinations. Under the electron microscope, the tumor cells demonstrated prominent nuclei and abundant mitochondria with no significant breakage (data not shown).

The higher tumor uptake of ^{99m}Tc-glucarate is not simply due to the distribution of the blood pool radioactivity and enhanced tumor vasculature with increase blood supply. The tumor blood pool activity in BT20 breast tumors bearing SCID mice has been compared with ^{99m}Tc-glucarate and ^{99m}Tc-DTPA uptakes. The tumor uptake was significantly greater with ^{99m}Tc-glucarate than with ^{99m}Tc-DTPA. ^{99m}Tc-DTPA enabled imaging the early tumor blood pool with ^{99m}Tc-DTPA soon after injection, but not at 3 hours post ^{99m}Tc-DTPA injection.

^{99m}Tc-sestamibi and ^{99m}Tc-glucarate exhibit different washout kinetics from the xenografted tumor models, as they are chemically different radiopharmaceuticals with different biological pathways. The rapid early clearance phase mainly reflects blood perfusion and effusion, in which the t_{1/2} values did not differ between ^{99m}Tc-glucarate and ^{99m}Tc-sestamibi in the MCF7/S tumors. The slow second phase, which reflects cellular efflux of radiotracers, showed shorter t_{1/2} in ^{99m}Tc-glucarate than that in ^{99m}Tc-sestamibi. The greater washout property of ^{99m}Tc-glucarate may be a disadvantage for tumor targeting. However, the larger amount of ^{99m}Tc-glucarate radioactivity initially delivered to the tumor might provoke the accumulation of ^{99m}Tc-glucarate continually higher than ^{99m}Tc-sestamibi as evidenced by tumor biodistribution data in the present study. Either in early phase or later phase washout of ^{99m}Tc-glucarate, there was no difference in t_{1/2} between the MCF7/S and MCF7/D40 tumors. This kinetic result demonstrates ^{99m}Tc-glucarate can be used as an agent in imaging breast tumors, including those with drug-resistant properties. The shorter half-time or faster washout in both of the early and second phase for ^{99m}Tc-sestamibi was observed in the MCF7/D40 tumors compared to the MCF7/S tumors. The faster washout of ^{99m}Tc-sestamibi from the drug-resistant tumors can thereby be used as a means of identifying MDR-Pgp expression in the patients with breast tumors.

The results show that ^{99m}Tc-glucarate localizes to resistant tumors and advantageously has significantly lower activity in soft tissue. The mean tumor-to-muscle ratio for ^{99m}Tc-glucarate is 4.5 and 6.2 (9.4 and 19.2 times higher than that for ^{99m}Tc-sestamibi) in the drug-sensitive tumors and drug-resistant tumors. In the BT20 tumor bearing mice, others have observed tumor to non-tumor (shoulder region tissue) was 20.53:1 twelve hours after intravenous injection of ^{99m}Tc-glucarate. This favorable tumor targeting property of ^{99m}Tc-glucarate may make this agent potentially useful to determine a small malignant lesion. Since ^{99m}Tc-sestamibi is not accurate in the detection of axillary lymph node metastasis, the visualization of malignant axillary adenopathy with ^{99m}Tc-glucarate might be superior to ^{99m}Tc-sestamibi. ^{99m}Tc-glucarate may provide utility visualizing breast tumors and axillary metastases in cases where the MDR Pgp is overexpressed or the tumors become resistant to drugs during chemotherapy.

The results of this example demonstrate that MCF7 human breast tumor xenografts can be detected by ^{99m}Tc-glucarate in vivo imaging with unequivocal visualization within 10-30 minutes, and remain visible for at least two hours after intravenous administration. ^{99m}Tc-glucarate offers favorable imaging properties of higher uptake in the MCF7 breast tumors compared to ^{99m}Tc-sestamibi.

Based on the results of this example, it is reasonable to administer a glucarate containing radiopharmaceutical imaging agent like ^{99m}T-glucarate to mammals, including humans, that have been characterized as expressing ABC transporter proteins. It is reasonable to expect based on the results of this example that the imaging agent would localize to one or more drug resistant tumors in the mammal. Based on the results of this example, it is also reasonable to expect that ^{99m}T-glucarate would localize to drug-resistant breast tumors, drug resistant lung tumors, and drug resistant myeloma. Based on the results of this example, it is also reasonable to expect that ^{99m}T-glucarate can localize to drug-resistant breast tumors in humans. Drug resistant tumors expressing ABC transporter proteins in mammals, including humans are expected to expel ^{99m}Tc-sestamibi due to the transport out of the cells in the tumor by Pgp, a drug-efflux pump. The results of this example illustrates the utility ^{99m}Tc-glucarate for detecting the location and extent of drug resistant tumors, and may find use imaging suspicious treatment resistant lesions, detecting axillary lymph node metastasis and evaluating various antitumor therapies.

### EXAMPLE 2

This example illustrates that the amount of ischemic reperfusion preconditioning or the amount of myocardial damage in the hearts with ischemia-reperfusion can be associated or correlated with myocardial accumulation levels of ^{99m}Tc-GLA as determined by region of interest and hot spot imaging. This example also illustrates that the degree or amount of ischemic preconditioning provided by a pharmaceutical agent may be characterized or correlated with myocardial accumulation levels of ^{99m}Tc-GLA as determined by region of interest and hot spot imaging and used to characterize or select a preconditioning pharmaceutical agent.

It is not known whether this association is reflected in the degree of myocardial injury in the ischemic-reperfused hearts. The severity of a myocardial injury can be estimated by the size of infarct or amount of irreversible myocytic necrosis. In clinical patients, infarct size can be determined with several noninvasive techniques, which has significance for general prognosis in patients after acute heart attack. It is known that ^{99m}Tc-GLA is accumulated by acute necrotic myocardium only. Using well-defined in vivo rat heart models, images of ^{99m}Tc-GLA localized to the models were obtained with a high-resolution stationary SPECT system called FASTSPECT. Ischemia-reperfusion injury of these models tissues were also analyzed by triphenyltetrazolium chloride (TTC) staining and electron microscope examination. The infarct size shown by ^{99m}Tc-GLA imaging was quantified and compared with the TTC staining analysis. Second, since the uptake and washout kinetics of viability dependent imaging agents are altered by the extent and severity of cellular and extracellular structural changes of ischemic-reperfused myocardium, the present study was undertaken to determine the in vivo kinetic properties of ^{99m}Tc-GLA for assessing the severity of myocardial ischemia-reperfusion injury in rat heart models exposed to different durations of left coronary artery (LCA) occlusion with reperfusion. Dynamic tomographic images were obtained in the beating rat model hearts with no motion of detectors or imaging aperture. These images were used to visualize the heart with ischemia-reperfusion injury and investigate the kinetics of cardiovascular imaging agents in small animal in vivo models.

Ischemic-Reperfused Rat Heart Models were prepare from Male Sprague-Dawley rats (weight, 250-300 g) that were anesthetized with 1.0%-1.5% isoflurane. The model of myocardial ischemia-reperfusion was developed using know techniques. The rat was prepared by first removing the fur from the chest and neck region with electric clippers. The jugular vein was catheterized through a surgical procedure. An incision was made over the jugular furrow, and blunt and sharp dissection was used to isolate the jugular vein. A PE-10 catheter was advanced through a nick made by micro scissors and then secured by ligatures. After tracheotomy, the rats were ventilated using a volume-controlled Inspira Advanced Safety Ventilator (Harvard Apparatus, Inc.) with a mixture of oxygen and room air. Using each animal's weight, the tidal volume (1.2 mL/100 g) and respiration rate (65-70 breaths/min) were automatically calculated in Safe Range settings (Harvard Apparatus). With a left intercostals thoracotomy incision, the chest was opened at the fourth or fifth intercostal space. The pericardium was opened and the heart was exposed. A small, curved, tapered needle threaded 5-0 silk through the tissue between the pulmonary cone and the left auricular appendage. A ligature was placed around the LCA with a small amount of myocardium. The ligature was pulled tight by passing the suture through a polyethylene tubing and clamping it for coronary occlusion. After the LCA was occluded for 30 or 90 min, 30 min of reperfusion was achieved by releasing the ligature. Hydrochloride lidocaine (5%) was administered via intravenous injection and cardiac surface drop when ventricular fibrillation was induced by LCA occlusion or reflow. During the period of surgery and ischemia-reperfusion treatment, the body temperature of the animal was maintained with a heating pad. The chest of the rat was closed before the animal was imaged with FASTSPECT.

Eighteen rats were divided into 2 groups to receive regional myocardial ischemia-reperfusion treatments. Group 1 animals (IR30, n=12 [IR=ischemia-reperfusion]) were treated by ligating the LCA for 30 min and releasing the ligature for 30 min. The LCA in group 2 animals (IR90, n=6) was ligated for 90 min and then released for 30 min. The experimental protocols are showed in FIG. 8.

Radiopharmaceutical preparation; GLA kits were provided by Molecular Targeting Technologies Inc. The reagent in the vial was a sterile, nonpyrogenic, lyophilized composition of potassium chloride, GLA, sodium bicarbonate, and hydrochloric acid. A vial of GLA was reconstituted by the addition of 1 mL of ^{99m}Tc as sodium pertechnetate (no <2.59 GBq [no <70 mCi]) in accordance with the manufacturer's instructions. The solution in the vial was allowed to stand at room temperature for 15 min. ^{99m}Tc-GLA radiochemical purity (RCP) was verified by thin-layer liquid chromatography using Gelman instant thin-layer silica gel (ITLC-SG) strips developed in saline and acetone. In the ITLC-SG strip developed by saline, ^{99m}Tc-colloid remained at the origin while ^{99m}Tc-GLA and ^{99m}Tc-pertechnetate migrated near the solvent front. In the strip developed by acetone, ^{99m}Tc-GLA and ^{99m}Tc-colloid remained at the origin while ^{99m}Tc-pertechnetate moved near the solvent front. The RCP of ^{99m}Tc-GLA exceeded 95% for all experimental injections. ^{99m}Tc-GLA was used within 3 h after preparation.

The high-resolution stationary SPECT system, FASTSPECT, was built in the Department of Radiology Research Laboratory at The University of Arizona (as described by references 9-11 in Liu et. al, J. Nucl. Med. (2004), vol. 45, pp 1251-1259). In this system, 24 small modular γ cameras are arranged in 2 circular arrays with a cylindric aperture. Each modular camera possesses a 10×10 cm Rexon NaI(Tl) scintillation crystal (Rexon Components, Inc.), an optical light guide, 4 square (5×5 cm) Hamamatsu Photonics photomultiplier tubes, and driver/amplifier electronics. Twenty-four 1-mm diameter pinholes were drilled in the aperture such that a point source in the center of the field of view (FOV) is simultaneously projected to the center of each camera. The dead space between adjacent cameras at the director surface is 7% of the total area. The total magnification is 3.5 in a 3.0×3.2×3.2 cm FOV. The spatial resolution of the system is about 1.0 mm with a sensitivity of 0.359 cps/kBq (13.3 cps/μCi) in air.

The rat was placed inside the aperture using a 6-cm-diameter cylindric cardboard holder mounted on a translation stage. The animal was positioned so that the heart localized in the center of the FOV. Thirty minutes after LCA reflow, ^{99m}Tc-GLA (148-296 MBq [4-8 mCi]) was injected as a bolus via the jugular vein catheter, followed by a 0.2-mL saline flush. Dynamic images were acquired every 1 min for 10 min beginning immediately on injection, followed by 5-min acquisitions at 15, 20, and 30 min. Then 5-min images were obtained every 15 min from 30 to 120 min after injection. A total of 24 projections were obtained, one from each camera. Each projection image was formed using a 24-bit word as an entry to a precomputed look-up table scheme. In this scheme, each scintillation event within the Nal crystal of the camera was registered as the digitized outputs from the camera's 4 photomultiplier tubes. To estimate energy and interaction position, the 4 outputs were then compared with the 20-bit look-up table. This table was precalculated using a calibration procedure that involved moving a collimated source across the camera face.

The maximum-likelihood expectation-maximization reconstruction algorithm was applied to generate 3D images. All images were reconstructed using 100 iterations. Using SlicerDicer software (PIXOTEC LLC), 3D images were computed to provide images in a 33 49 49 voxels format, reoriented manually regarding the cardiac axis. Then serial tomographic short-axis, vertical, and horizontal long-axis slices of the heart with 1-pixel thickness (1.0 mm) were generated.

A region of interest (ROI) was established over a hot spot with the highest activity of ^{99m}Tc-GLA (counts/pixel) selected from a short-axis slice of the 120-min image. The ROI was applied to all images from 1 to 120 min after injection. After correction for background and ^{99m}Tc decay, the radioactivity (counts/pixel/min) in the hot spot was projected onto the dynamic images to determine the time-activity curve. Using an ROI selected from the non-LCA region (left ventricular septum), the normal myocardial time-activity curve was generated and compared with the curve of the infarct zone. Hot spots on all short-axis slices of 120-min image were quantified with ROI analysis to calculate the ratios of necrotic zone activities to normal myocardial activities and averaged over the whole heart to produce a mean ratio.

Measurement of Myocardial Risk and Infarct. After imaging acquisition was completed, the animal was moved out of the aperture. The chest of the rat was reopened and the LCA was reoccluded using the same ligature. To determine the myocardial ischemic area at risk (IAR), 1.0 mL Evans blue (20%) in phosphate-buffered saline (PBS) was injected through the femoral vein, allowing dye to stain the nonischemic portion of the heart. The risk zone was identified as that region lacking blue staining. An overdose injection of pentobarbital was followed immediately to kill the animal. The entire heart was expeditiously excised, weighed, and rinsed of excess dye with cold saline. ^{99m}Tc activity in the heart was measured in a CRC-4 radioisotope dose calibrator (Capintec). The great vessels, atria, and right ventricle of the heart were removed. The left ventricle was sectioned into 4 transverse slices in a plane parallel to the atrioventricular groove. Both sides of each tissue slice were photographed immediately using a D-500L digital camera (Olympus Optical Co.).

TTC staining was used to identify the area with infarct. The tissue slices were incubated in 1% TTC PBS solution (pH 7.4) at 37° C. for 20 min and subsequently fixed in 10% PBS-buffered formalin overnight at 2° C.-8° C. The viable myocardium stained by TTC was dark red. Both sides of each TTC-stained tissue slice were photographed again with the digital camera.

The IAR (unstained by Evans blue dye) and the TTC-negative area (white or pale, necrotic myocardium) on the digital photographs were outlined and quantified using the SigmaScan software (SPSS Science) in trace-measurement mode. Infarct size was expressed both as a percentage of total LV mass (% LV) and as a percentage of the IAR (% IAR).

Determination of Infarct on Imaging. The hot spot size of ^{99m}Tc-GLA on the 120-min image was determined on short-axis slices using the trace-measurement mode of SigmaScan software as described. The left ventricular wall was outlined using the 1-min cardiac blood-pool image as a contrast. The size of hot spots on short-axis slices were averaged as a percentage of the entire left ventricle and then normalized to the percentage of the IAR (% IAR).

Assessment of Ultrastructural Injury. An additional 4 rats were subjected to the experimental protocol without radiotracer administration. Two rat hearts were treated as the IR30 protocol in group 1; others were treated as the IR90 protocol in group 2. After a 30-min reperfusion, 0.5 mL saline was intravenously injected to match the injected volume of ^{99m}Tc-GLA and saline in group 1 and group 2. The anesthetized rats were kept alive under the same experimental conditions as described for 2 hours and then killed at the end of the experiment. Two small blocks of left ventricular tissue were selected from the LCA ischemic-reperfused central area and nonischemic area (septum) of each heart, respectively. The tissue blocks were immersed in a phosphate-buffered 3% glutaraldehyde solution (pH 7.4) for 3 h at 4° C. Then the samples were transferred and postfixed in 1% osmium tetroxide, dehydrated in a graded series of ethanol, and embedded in epoxy resin. Ultrathin sections were stained with uranyl acetate and lead hydroxide and examined with a transmission electron microscope ([TEM] Phillips CM12). All results are expressed as mean±SEM. Comparisons between groups were performed with 1-way ANOVA. Comparisons between 2 variables within a group were made using a paired t test. Probability values 0.05 were considered significant. The correlation between myocardial ^{99m}Tc-GLA hot spots and myocardial infarct size as measured by TTC stain was assessed by linear regression analysis.

Hot spot and normal zone activities were normalized to peak activities at 2 min for each experiment and averaged for the group to produce mean myocardial time-activity washout curves. The kinetic washout curves of ^{99m}Tc-GLA were fit with nonlinear regression procedures using TableCurve 2-dimensional software (Systat Software, Inc.). All experiments were performed in accordance with the animal research guidelines of the National Research Council and were approved by the Institutional Animal Care and Use Committee at The University of Arizona.

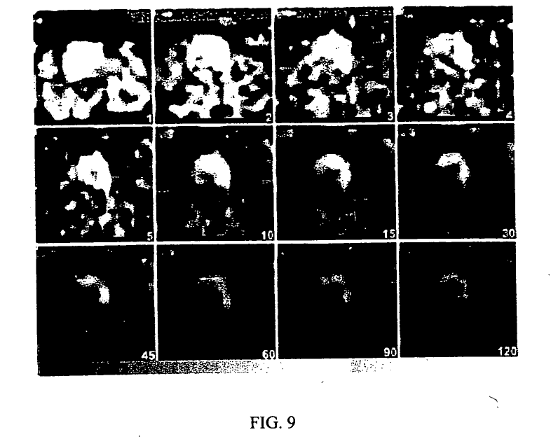

A representative reconstructed 3D display of 120-min FASTSPECT images of a rat heart subjected to the IR90 protocol was made and showed that the wall of the left ventricle was partly visualized in the stenosis zone of LCA supplied area. Selected tomographic short-axis, horizontal, and vertical long-axis slices of the heart were also constructed and exhibit a hot spot (increased ^{99m}Tc-GLA uptake) localized on the anterior wall, lateral wall, and apex of the left ventricle. Identical hot spots were detected in all rat hearts of group 1 and group 2 with ischemia-reperfusion imaged with ^{99m}Tc-GLA. A good infarct definition with the regional hot spot could be achieved 10 min after intravenous administration. The infarct remained well defined for at least 120 min after injection. FIG. 9 demonstrates representative ^{99m}Tc-GLA in vivo FASTSPECT dynamic images in an IR30 heart.

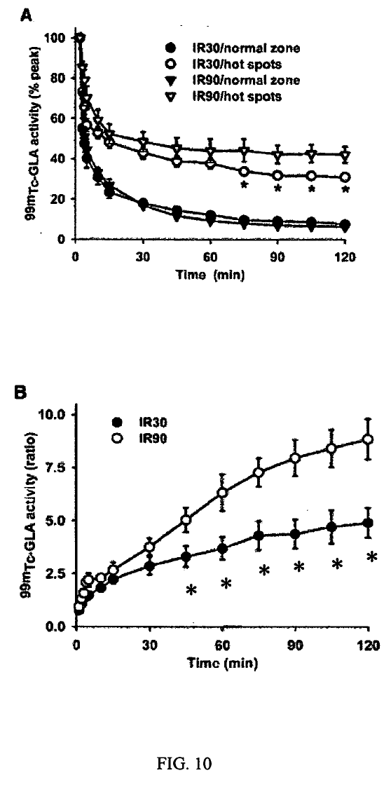

Quantitative Analysis of ^{99m}Tc-GLA Imaging. Myocardial ^{99m}Tc-GLA activities were quantified with the computerized ROI analysis of FASTSPECT images that were background-subtracted and decay-corrected. FIG. 10A shows normalized myocardial time-activity washout curves. ^{99m}Tc-GLA exhibited biphasic washout from the LCA hot spot and normal myocardial zone. The kinetic washout curves were plotted from 2 to 120 min with a significant difference observed at each point in time from 10 to 120 min between the LCA hot spot and normal zone in group 1 and group 2. Beginning at 75 min, the normalized hot spot activity in group 2 was significantly higher than that in group 1. Total 2-h fractional washout from the hot spot was significantly lower than that of the normal zone. As a result, the fractional retention (% peak) was significantly higher in the hot spot than that in the normal zone (31.0±1.3 vs. 7.5±1.0 in IR30, P<0.05; 42.1±4.0 vs. 6.4±0.9 in IR90, P<0.05) by the end of experiment. The hot spot of group 2 exhibited significantly higher ^{99m}Tc-GLA retention than that of group 1 (P<0.05). By fitting each individual curve using the TableCurve calculation, biexponential equations were found to provide the best fits to the curves. The early phase showed fast washout and the late phase showed slow washout. The best fit equation was: y=a exp(−bx)+c exp(−dx). There was no significant difference in the half-time value (t_{1/2}) for the early phase between the hot spot and normal zone (IR30: 2.4±0.4 vs. 1.6±0.4, P<0.05; IR90: 3.8±1.1 vs. 2.0±0.2, P<0.05). However, there was a significant difference (P<0.05) for the t_{1/2} in the late phase (IR30: 216.3±23.8 vs. 66.6±5.5, P<0.05; IR90: 511.5±74.2 vs. 49.1±2.9, P<0.05).

FIG. 10B shows that the ratios of the hot spots to normal myocardial activities increased progressively after ^{99m}Tc-GLA administration in the IR30 and IR90 hearts. Beginning at 45 min, the ratio in group 2 was significantly higher than that in group 1. The final ratio was 4.91±0.71 in group 1 and 9.53±1.76 in group 2 (P<0.05). The ratio in group 1 with the IR30 protocol was 1.94-fold lower than that in group 2 with the IR90 protocol. Two hours after injection, the averaged ratio of the whole-heart hot spot activity to normal myocardial activity was 4.1±0.3 in group 1 and 7.1±1.1 in group 2 (P<0.05).

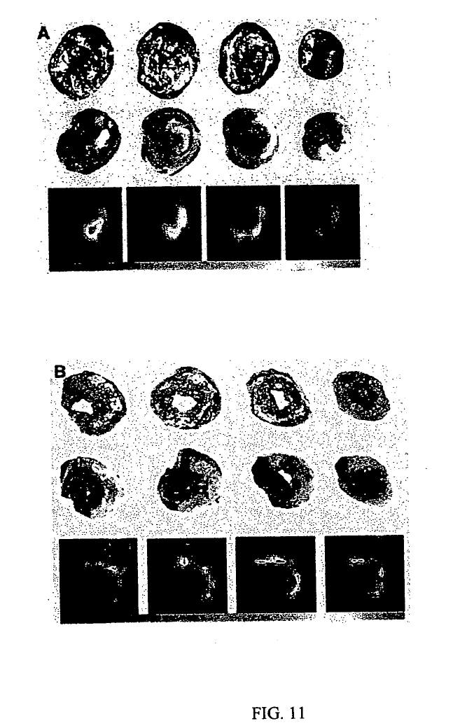

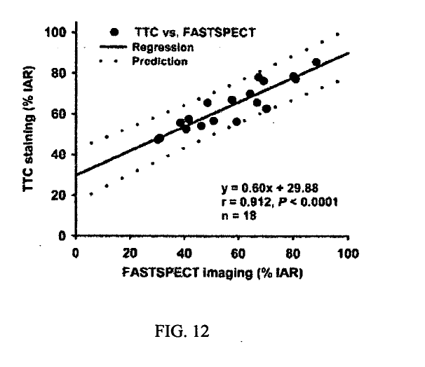

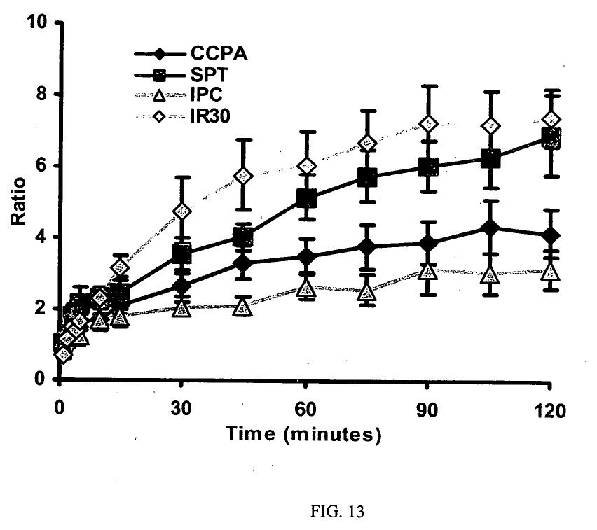

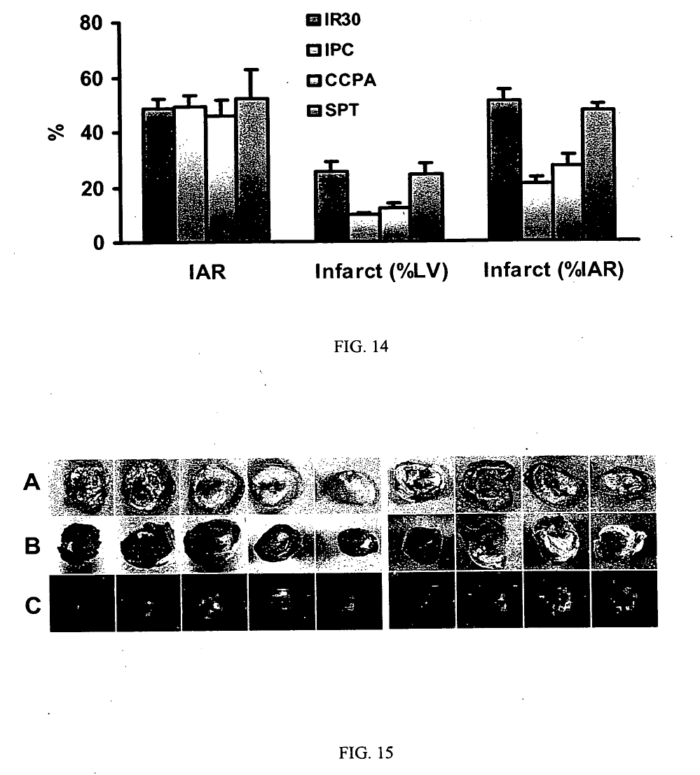

Myocardial IAR and Infarct Size. The measurements of myocardial ischemic IAR and infarct are shown in Table 3. The difference of myocardial ischemic IARs as shown by lack of Evans blue between the hearts subjected to 30-min and 90-min LCA occlusion was not significant. The ^{99m}Tc-GLA hot spots on FASTSPECT images were consistent with the unstained areas on TTC staining (FIGS. 11A and 11B). The percent infarct size of left ventricles measured by TTC staining in group 1 was significantly smaller than that of group 2. Normalized by the ischemic IAR, the infarct size (% IAR) in group 1 was still significantly smaller than that of group 2. The quantified hot spots with ^{99m}Tc-GLA on FASTSPECT imaging represented a good agreement with the infarct measurements on TTC staining. The hearts subjected to 90-min LCA occlusion exhibited significantly larger hot spots compared with the hearts subjected to 30-min LCA occlusion (P<0.05). There was a significant correlation between the infarct measurements (corrected by IAR, % IAR) with TTC staining and ^{99m}Tc-GLA quantified hot spot imaging in either group 1 (r=0.797, P=0.0019) or group 2 (r=0.956, P=0.0029). FIG. 12 illustrates the overall correlation between the measurements in all 18 experimental hearts (r=0.912, P<0.0001).

Representative electron photomicrographs made of thin sections of myocardial tissue samples for group 1 and group 2 were taken. There were no significant TEM morphologic differences noted among sections taken from the septa of the left ventricles of the hearts subjected to 30-min or 90-min LCA occlusion followed by reperfusion. These samples exhibited a normal morphologic appearance. In contrast, hearts subjected to the ischemia-reperfusion treatment showed abnormalities in mitochondrial and myofilament morphology with irreversible myocytic injury indicated by electron-dense inclusions and membrane breaks. In contrast to the 30-min ischemia heart, the 90-min ischemia heart exhibited evidence of more severe, irreversible injury, including breaks in sarcolemma, abnormal nucleus, hyperplasia of mitochondria, and dense intramitochondrial granules. Matrix dilution, swelling, fenestration, and paucity of cristae with loss of cytosolic glycogen were found in most mitochondria.

TABLE 3
Myocardial IAR and Infarct Measurements

| Parameter | IAR (% LV) | TTC (% LV) | TTC (% IAR) | GLA (% LV) | GLA (% IAR) |
|---|---|---|---|---|---|
| IR30 (n = 12) | 48.8 ± 3.2 | 24.5 ± 3.2 | 49.2 ± 4.3 | 28.4 ± 2.1 | 58.4 ± 2.7 |
| IR90 (n = 6) | 55.2 ± 1.8 | 40.8 ± 3.6* | 73.4 ± 4.7 | 42.0 ± 2.6* | 75.9 ± 2.7* |

*P < 0.05 compared with IR30;
% LV = % LV mass;
Data are expressed as mean ± SEM.

In this example, dynamic high-resolution SPECT imaging enabled detection of myocardial hot spots in rat heart models exposed to 30-min and 90-min LCA occlusion followed by reperfusion with ^{99m}Tc-GLA. Ischemic-reperfused treatments in the 2 models produced an average infarct size of 24.5% and 40.8% as measured by TTC staining, respectively, with a significant difference. The difference in severity of ischemia-reperfusion injury between the 2 rat heart models was confirmed by ultrastructural electron microscope examination. Dynamic high-resolution SPECT imaging of ^{99m}Tc-GLA localized in the heart models demonstrated a good infarct definition 10 min after injection. The infarct remained well defined for at least 120 min after injection. The ratio of the hot spot to normal myocardial activity increased progressively after ^{99m}Tc-GLA administration. Beginning at 45 min after imaging agent injection, the ratios in hearts subjected to 90-min LCA occlusion and reperfusion were significantly greater than that in hearts subjected to 30-min LCA occlusion. The slower kinetic washout of ^{99m}Tc-GLA in the IR90 hearts resulted in a significantly higher radioactive retention compared with that of the IR30 hearts. The hot spots of the IR90 hearts determined with ^{99m}Tc-GLA dynamic high-resolution SPECT imaging, which represented a good agreement with the infarct measurements on TTC staining, were significantly larger relative to the hot spots in IR30 hearts. These results confirm that ^{99m}Tc-GLA is preferentially retained in acute necrotic myocardium after onset of coronary occlusion. More important, the amount of retention of ^{99m}Tc-GLA and the size of the hot spot in ischemic-reperfused myocardium are correlated with the severity of myocardial injury.

^{99m}Tc-GLA has been tested in a variety of animal models to investigate its infarct-avid localizing properties, including ex vivo isolated perfused rat and rabbit hearts and in vivo rabbit, canine, and swine heart models. Using a swine model with myocardial ischemia induced by a catheter-mounted stenosis in the left anterior descending coronary artery, positive uptake of ^{99m}Tc-GLA in the severe ischemic myocardium has been reported. Of the 15 ^{99m}Tc-GLA scan-positive studies, 8 animals showed scattered TTC-negative staining in the IAR. Electron microscope analysis demonstrated focal irreversible myocyte injury characterized by flocculent densities within mitochondria from the risk region with ^{99m}Tc-GLA scanpositive and TTC-positive staining. ^{99m}Tc-GLA was sensitive enough to be able to detect early mild scattered myocardial infarcts and micronecroses. Obviously, TTC staining might not delineate myocyte injury in detail and neglect micronecroses. In this example the regional myocardial injury in the ischemic-reperfused rat heart models was assessed using both TTC staining and TEM analysis.

Previous workers used a 15-min global ischemia with a complete reperfusion rat heart model in comparison with 90-min ischemia. Significantly higher myocardial retention of ^{99m}Tc-GLA was found in the 90-min ischemia model but not in the 15-min ischemia model. Effluent creatine kinase (CK) measurement demonstrated that 90 min of flow interruption was sufficient to induce myocardial necrosis, whereas 15 min was not. It was reported that the TTC-negative area might contain approximately 13% reversible injury in an LCA occlusion rat model with 20-min ischemia. After 20 min of ischemia, myocardial infarct is markedly increased and further exacerbated by reperfusion. With 30 min of coronary occlusion, the necrotic area can be clearly delineated within the myocardium with the TTC staining technique.

The in vivo kinetic properties of ^{99m}Tc-GLA for assessing the severity of myocardial ischemia-reperfusion injury in this example utilized rat heart models with 30-min and 90-min LCA occlusion followed by reperfusion and compared the severities of irreversible necrosis. As a result, the radioactivities and sizes of ^{99m}Tc-GLA hot spots were significantly distinguishable between the hearts subjected to different durations of ischemia. The high avidity of ^{99m}Tc-GLA for the infarct makes its uptake visualizable within 10 min after intravenous administration. Forty-five minutes after injection, the ratios of hot spots to normal zone radioactivities showed a significant difference between the 30-min and 90-min ischemia heart models.

The results of this example show that 30 min of LCA occlusion followed by a 30-min reperfusion induces a significantly slower washout of ^{99m}Tc-GLA in the necrotic myocardium compared with that of the normal myocardial zone. The kinetic alteration of ^{99m}Tc-GLA is markedly accelerated by 90-min LCA occlusion. A biexponential function was found to be the best fit for describing the washout pattern of ^{99m}Tc-GLA. A rapid early clearance phase followed by a slow second phase was observed in the ischemic-reperfused myocardium and normal zone. In the early phase, which mainly reflects blood perfusion and effusion, the t_{1/2} of ^{99m}Tc-GLA from the biexponential function did not differ between the infarct and normal zones. In the second phase, which reflects cellular efflux of ^{99m}Tc-GLA, the t_{1/2} of the hot spot was significantly longer than that in the normal myocardium. The principal differences among the hearts subjected to 30-min and 90-min ischemia resulted from a differential late phase washout. The longer t_{1/2} or slow clearance in the second phase was more pronounced in 90-min ischemic hearts. These data indicate that the severity of myocardial injury induced by different durations of ischemia with reperfusion in rat heart models can be quantitatively correlated with ^{99m}Tc-GLA hot spot imaging.

Previous work illustrated that the infarct size can be quantitatively measured in rat heart models with ^{99m}Tc-sestamibi defects or areas where the ^{99m}Tc-sestamibi does not accumulate. ^{99m}Tc-Sestamibi myocardial activity correlates the amount of viable myocardium stained by TTC. The extent of the left ventricular perfusion defect on ^{99m}Tc-sestamibi images reflects the size of the myocardial infarction. Advantageously, ^{99m}Tc-GLA is accumulated by infracted tissue only. The results of this example indicate that hot spot imaging with ^{99m}Tc-GLA can be used for the determination of infarct size in persistently occluded or reperfused myocardial infarcts. Measurement of ^{99m}Tc-GLA activity quantified with ROI analysis of images that were background subtracted and decay corrected demonstrated that the hot spot size of ^{99m}Tc-GLA correlates well with the infarct size determined by TTC staining. In comparison with ^{99m}Tc-sestambi imaging, in which the defects are clearly contrasted with normal myocardium, negative accumulation of ^{99m}Tc-GLA in normal myocardium makes the quantitative analysis difficult to calculate the percentage of infarct over the whole left ventricle in the heart with a small size infarct. The early blood-pool image was used to identify the left ventricular wall range so that the percentage of infarct size could be quantified in the present example. Using a computed 3D display, absolute infarct volume may be measured more reliably, which provides insight into prognosis after acute myocardial infarction.

The in vivo kinetics of cardiovascular radiopharmaceuticals with dynamic high-resolution SPECT imaging in rat heart models was determined in this example. Large mammals, such as canines, are often used for in vivo experimental imaging in coronary artery disease. However, the cost for large mammals is high, and experience with surgical techniques is required. The small-mammal heart model, such as rodents, offers advantages over large-animal or mammal models, such as low cost, simple procedures in surgery, and a high successful rate in modeling. In addition, many gene therapy strategies for the treatment of cardiac failure and long-term myocardial protection are being studied in rodent models with noninvasive imaging requirement. In recent years, high-resolution tomographic imaging systems have become available for small-animal studies in basic biomedical science so that many experimental procedures in large animal cardiac models can be performed in rat models. In the rat heart models of the present example, dynamic cardiac images were acquired with an imaging system using procedures similar to those required by clinical physicians. Myocardial radioactivity in the present example could be quantified accurately and the washout kinetics of ^{99m}Tc-GLA were determined effectively. The quantitative analysis of myocardial infarct on tomographic images showed a significant correlation with biochemical staining measurement.

The results of this example demonstrate the property of ^{99m}Tc-GLA to positively identify acute infarct myocardium. ^{99m}Tc-GLA can mark nonviable regions by hot spot imaging in myocardium with infarct. Therefore, the severity of myocardial injury induced by different durations of ischemia after reperfusion could be assessed noninvasively and quantitatively. The results show that there is a correlation between the degree or extent of a myocardial infarct or injury and the size of the hot spot. This agent would appear to be clinically useful to determine the location and size of acute infarcted myocardium very early. ^{99m}Tc-GLA imaging may not only provide an imaging tool to diagnose equivocal myocardial infarct in patients with heart attacks, allowing differentiation of acute from recent infarcts, but also direct the use of thrombolytic therapy. Quantitative analyses on dynamic images with ^{99m}Tc-GLA would make it possible to identify myocardial acute necrosis earlier and more accurately and provide a unique, noninvasive tool for evaluation of patient prognosis.

### EXAMPLE 3

In this example the properties of ^{99m}Tc-GLA in detecting xenografted human breast-tumor with multidrug resistance is shown. The in vivo kinetics of ^{99m}Tc-GLA with ^{99m}Tc-MIBI in SCID mice by imaging using a high-resolution SPECT system is also shown.

^{99m}Tc-sestamibi (MIBI) scintimammography has been shown to have clinical utility in identifying breast tumors. However, some breast tumors are difficult to detect by MIBI imaging due to a multidrug resistance (MDR) gene, which encodes for a membrane P-glycoprotein (Pgp). Pgp acts as an energy-dependent drug-efflux pump and allows transport of structurally and functionally unrelated drugs out of cells. ^{99m}Tc-MIBI as a surrogate marker of Pgp function also can be pumped out of tumor cells with Pgp expression.

D-glucaric acid (glucarate) is a six-carbon dicarboxylic acid sugar, which can be radiolabeled with ^{99m}Tc resulting in ^{99m}Tc-glucarate (^{99m}Tc-GLA). ^{99m}Tc-GLA may be an agent for detecting breast tumors. In drug sensitive breast tumor xenografts, ^{99m}Tc-GLA was found to have significantly higher uptake than ^{99m}Tc-MIBI.

Tumor cell lines used. MCF7/S: Cells are parental, drug (doxorubicin) sensitive, breast carcinoma cells. MCF7/D40: Cells were generated in vitro by successive culturing parental MCF7 cells in slowly increasing concentrations of doxorubicin with a total selection time of 31 months. Twenty-eight SCID mice were studied. MCF7/S or MCF7/D40 tumors were grown for 10-14 days after subcutaneous injection of 1×10⁶ cells into the right thigh. Groups of mice with the xenografted tumors included: (MCF7/S group: MIBI: n=9; GLA: n=8) and (MCF7/D40 group: MIBI: n=6; GLA: n=5).

The dynamic imaging system SPECT consisted of 24 small modular gamma cameras. Each modular camera consists of a 10 cm×10 cm NaI(Tl) scintillation crystal, 4 square (5 cm×5 cm) Hamamatsu photomultiplier tubes. The system included a cylindrical aperture with twenty four 1-mm diameter pinholes drilled in the aperture. A point source in the center of the field of view is projected simultaneously to the center of each camera. Total magnification is 3.5 in a 3.0 cm×3.2 cm×3.2 cm field of view. The spatial resolution of the system is about 1.0 mm, with a sensitivity of 13.3 cps/μCi.

^{99m}Tc-GLA or ^{99m}Tc-MIBI (5-8 mCi, 185-296 MBq) was injected via a jugular vein catheter. Dynamic images were acquired for 2 hours. Twenty-four projections were obtained, one from each camera. Each scintillation event within the camera's NaI crystal was registered as the digitized outputs from the camera's four photomultiplier tubes. The four photomultiplier outputs were compared to a 20-bit look-up table derived from a calibration procedure that involved moving a collimated source across the camera face.

Tumor image reconstruction was performed using 100 iterations of the maximum-likelihood expectation-maximization (ML-EM) algorithm. Using SlicerDicer software, three-dimensional images were computed to provide images in a 33×49×49 voxels format, and generate tomographic transaxial, coronal and sagittal slices with one-pixel thickness (1.0 mm). Regions of interest (ROI) over the tumors were created and applied to the dynamic images for determining time-activity curves. The percent washout rate relative to the peak uptake of the tumors were calculated.

Biodistribution and Western blot analysis were performed and the results given in Table 4. Tumors and organs were harvested and counted in a dose calibrator. The percent injected dose per gram (% ID/g) was calculated. Membrane preparations made from MCF7/S and MCF7/D40 tumors were immunoblotted with the mouse monoclonal antibody C219 against Pgp followed by a secondary antibody, which was detected by chemiluminescence. Western Blotting analysis showed that MCF7/D40 are Pgp positive and 40-fold resistant to doxorubicin compared to MCF7/S. Expression of Pgp in the MCF7/D40 cells was demonstrated in Western blots of tumor cell membrane preparations. No immunodetectable MDR1 Pgp was presented in MCF7/S cells.

The results of this example show that Western blot analysis can be used to determine Pgp in the MCF7/D40 tumors and that. no immunodetectable MDR1 Pgp was presented in the MCF7/S tumors. The MCF7/S tumors could be detected from 2 min to 120 min after injection of ^{99m}Tc-MIBI using the image system and methodology. In contrast, the MCF7/D40 tumors could be localized only 2-3 min post injection of ^{99m}Tc-MIBI. All tumors of ^{99m}Tc-GLA groups were initially well visualized within 5 min by the image system and methodology after injection of ^{99m}Tc-GLA and remained detectable for at least 2 hrs. There was no difference in ^{99m}Tc-GLA uptake and retention between the MCF/S and MCF7/D40 tumors. The biodistribution data of GLA demonstrated significantly higher accumulation (% ID/gm) in the tumors compared to MIBI. Biodistribution data illustrated the higher tumor radioactivity for the ^{99m}Tc-GLA and higher tumor to non-tumor ratios for the drug resistant and drug sensitive tumors compared to ^{99m}Tc-MIBI as shown by the data in Table 4.

TABLE 4
Biodistribution Data

| | GLA(S) | GLA(D40) | MIBI(S) | MIBI(D40) |
|---|---|---|---|---|
| Tumor | 1.16 ± 0.17 | 1.39 ± 0.12 | 0.64 ± 0.07* | 0.27 ± 0.05*† |
| Blood | 1.19 ± 0.17 | 1.07 ± 0.14 | 0.06 ± 0.01* | 0.07 ± 0.01* |
| Muscle | 0.29 ± 0.02 | 0.25 ± 0.04 | 1.37 ± 0.12* | 1.08 ± 0.27 |
| Heart | 0.70 ± 0.09 | 0.55 ± 0.1 | 10.61 ± 0.89* | 9.84 ± 1.39 |
| Lung | 1.15 ± 0.11 | 1.41 ± 0.54 | 0.95 ± 0.16 | 0.61 ± 0.10 |
| Liver | 2.10 ± 0.35 | 1.56 ± 0.18 | 7.02 ± 1.01 | 8.29 ± 1.43* |
| Kidneys | 22.59 ± 1.38 | 22.10 ± 1.59 | 21.32 ± 2.03 | 19.63 ± 3.48 |
| Tumor/ | 1.13 ± 0.23 | 1.38 ± 0.19 | 13.97 ± 2.89* | 3.93 ± 0.41* |
| Blood | | | | |
| Tumor/ | 4.37 ± 0.89 | 6.23 ± 0.89 | 0.48 ± 0.05* | 0.31 ± 0.05* |
| Muscle | | | | |

The results in this study also demonstrate that MCF7 human breast tumor xenografts can be detected by ^{99m}Tc-glucarate in vivo imaging. ^{99m}Tc-glucarate offers favorable imaging properties of higher uptake and retention in the breast tumors compared to ^{99m}Tc-sestamibi. More importantly, ^{99m}T-glucarate can potentially detect the drug-resistant breast tumor ignored by ^{99m}Tc-sestamibi imaging.

The small-mammal xenografted tumor model, offers advantages over large-mammal models, such as low cost, simple procedures in surgery, and a high successful rate in modeling. In addition, many gene therapy strategies, drug regimens, and radiation therapies for the treatment of tumors and other drug resistant lesions may be studied and modified in long term in rodent models with noninvasive imaging requirement. In recent years, high-resolution tomographic imaging systems have become available for small-animal studies in basic biomedical science so that many experimental procedures in large animal cardiac models can be performed in rat models.

### EXAMPLE 4

This example illustrates characterizing cells and tissue that have been protected or made resistant to the effects of subsequent ischemia. The example further illustrates how the extent of the imaging agent in the tissue may be correlated with the extent of a course of ischemic preconditioning treatment. The extent of localization of the imaging agent, preferably ^{99m}Tc-GLA, may also be used to identify and develop compounds that provide ischemic precondition and also characterize the extent of protection provided by the compounds. The imaging agent and detection methodology can be used to determine whether adenosine A1 receptor is involved in protection induced by ischemic preconditioning in the rat heart. If the adenosine receptor is involved, an adenosine receptor like agonist, 2-chloro-N6-cyclopentyladenosine (CCPA), should give protection similar to ischemic preconditioning, while an A1 receptor antagonist, 8-(p-sulfophenyl)-theophylline (SPT), should block protection by ischemic preconditioning.

Myocardial ischemia-reperfusion injury can occur after angioplasty or thrombolysis and is more severe than transient myocardial stunning. It may be possible to modulate effects or myocardial ischemic-reperfusion injury using Ischemic preconditioning, Various drugs and chemicals may be used for preconditioning and myocardial ischemic preconditioning (IPC) whereby exposure of myocardium to a short period of non-damaging ischemic stress can lead to resistance to the deleterious effects of subsequent prolonged ischemic stress. Mild ischemia protects myocardium from effects of later ischemia The protection of this mild ischemia appears biphasic: early (2-3 hours) and late (up to 3 days). Pre-infarction angina is followed by smaller infarcts than in acute MI without angina. Preconditioning cardioprotection in Ischemia-Reperfusion Injury may be provided by ischemic preconditioning or chemical/pharmaceutical preconditioning using agents such adenosine, diazoxide, nitric oxide donors, or combinations of these.

Experimental groups in this example included: Group-I (IR30, n=11): Animals subjected to a treatment of 30-min regional myocardial ischemia and 30-min reperfusion by ligating and releasing the left coronary artery (LCA). Group-II: (IPC Group, n=6): Rats were preconditioned with a sequence of 6 cycles of 4-min LCA occlusion and 4-min re-flow; 10 min later, the rats underwent a 30-min LCA occlusion followed by 30-min re-flow. Group-III (CCPA, n=6): rat chests were opened for the equivalent time for IPC in Group-II. Ten minutes prior to 30-min LCA occlusion and 30-min reperfusion, CCPA (40 μg/kg) was intravenously injected with 5 cycles of 4-min infusion and 4-min no infusion. Group-IV (SPT, n=5): animals subjected to IPC as the protocol in Group-II, SPT (10 mg/kg) was intravenously infused 2-min before and during subsequent ischemia-reperfusion.

Treatments in this example consisted of control (IR30, n=8): no preconditioning; equivalent open-chest time; IPC (n=6): 4-min LCA occlusion, 4-min reflow, 5 cycles; CCPA (n=6): 4-min IV infusion, 4-min no infusion, 5 cycles; SPT (n=4): IPC first, SPT IV infusion 2 min before and during LCA occlusion and re-flow.

In this example, the high-resolution small-animal SPECT system consists of 24 small modular gamma cameras. Each modular camera consists of a 10 cm×10 cm NaI(Tl) scintillation crystal, 4 Hamamatsu photomultiplier tubes. A cylindrical aperture with 1-mm diameter pinholes. Total magnification is 3.5 in a 3.0 cm×3.2 cm×3.2 cm field of view. Spatial resolution of the system is about 1.0 mm, with a sensitivity of 12 cps/μCi.

Image acquisition and processing ^{99m}Tc-glucarate (185 MBq) was intravenously injected. Dynamic images were acquired every min for 30 min, followed by 5-min acquisition every 15 min until 2-hr. 24 projections were obtained, one from each camera. Reconstruction was performed using 100 iterations of the MLEM algorithm. Using SlicerDicer software, 3-D images were computed to provide images in a 33×49×49 voxels format, and generate transaxial, coronal and sagittal slices with one-pixel thickness (1.0 mm). Regions of interest (ROI) over the normal zone and ischemic-reperfused area were created and applied to the dynamic images for determining time-activity curves.

The results of the example show that dynamic ^{99m}Tc-glucarate imaging could assess IPC and adenosine effects in cardioprotection in rat heat models using a high-resolution SPECT system. Significant tolerance to myocardial ischemia-reperfusion injury, as assessed by biochemical assay and noninvasive infarct-avid imaging, was induced with an IPC protocol in the rat model. The cardioprotection of IPC could be simulated by adenosine receptor A1 agonist CCPA, or blocked by antagonist SPT. The results demonstrate that adenosine mediates protection by ischemic preconditioning in this specific rat heart model.

^{99m}Tc-glucarate imaging is not only useful in detecting early ischemia-reperfusion injury, but can also be used in evaluating the effects of cardioprotective treatments. Quantitative analyses on dynamic images with ^{99m}Tc-glucarate would make it possible to identify myocardial ischemia-reperfusion injury more accurate, and provide a unique tool for evaluation of cardioprotection. Using the system and methods in this example, imaging with the ischemic-reperfused rat heart model provides a solution-specific approach with high-resolution and fast dynamic acquisition for kinetic studies of new myocardial imaging agents and pharmaceutical agents for preconditioning.

### EXAMPLE 5

A patient complaining of invalidation pain in the spine (mid-thorax) for about two months. Peak of monoclonal IgG-k in serum. Bence-Jones protein in urine. Spine CT. lytic lesion of T8, with soft tissue infiltration. Bone biopsy: plasmocytoma (localized myeloma). ^{99m}Tc-MDP bone scan (posterior) showed focally increased uptake corresponding to T8. ^{99m}Tc-sestamibi scan (posterior) showed negative for bone marrow infiltration. Scintigraphy with ^{99m}Tc-GLA with sequential imaging times showed focally increase tracer uptake in the bone lesion and in soft tissue. Uptake was obvious in the early images, 15 and 30 min p.i., late acquisition, 6 hr p.i., made the tracer uptake much more obvious. After about 1 year of stabilization and external radiotherapy, ^{99m}Tc-MDP bone scan (posterior) showed inhomogeneous tracer distribution, without clear focally increased uptake. ^{99m}Tc-GLA (posterior view) showed normal physiologic tracer distribution, without areas of focally increased uptake.

Although the present invention has been described in considerable detail with reference to certain preferred embodiments thereof, other versions are possible. Therefore the spirit and scope of the appended claims should not be limited to the description and the preferred versions contain within this specification.

1. A method comprising the acts of:
imaging ^{99m}Tc-GLA localized in one or more resistant cells; and
providing additional treatment to the resistant cells based on the extent of the ^{99m}Tc-GLA localized in the cells.

2. The method of claim 1 where the ^{99m}Tc-GLA localized to a tissue or organ.

3. The method of claim 1 where the cells are resistant to prolonged ischemic stress.

4. The method of claim 1 where the cells are resistant to one or more chemotherapeutic agents.

5. The method of claim 1 where the one or more resistant cells are part of a drug resistant tumor.

6. The method of claim 1 where the one or more resistant cells are ischemic preconditioned myocardial cells.

7. The method of claim 1 where the additional treatment includes the act of administering one or more cytotoxic agents, reversing agents, or a combination of these to the resistant cells.

8. The method of claim 1 where the extent of the ^{99m}Tc-GLA localized in the cells is determined by acts including region of interest analysis.

9. A method for detecting a drug resistant tumor in a patient comprising the acts of:
administering to a patient with signs of a drug resistant tumor a detectable amount of a composition comprising ^{99m}Tc-GLA; and
detecting the extent of ^{99m}Tc-GLA localized in the drug resistant tumor in a patient.

10. The method of claim 8 further including the act of treating the drug resistant tumor following detection of the localized ^{99m}Tc-GLA.

11. The method of claim 8 where sign of a drug resistant tumor is detection of a cellular transporter protein.

12. The method of claim 8 where the drug resistant tumor is a drug resistant breast cancer tumor, a drug resistant lung cancer tumor, or a drug resistant myeloma.

13. The method of claim 8 where the drug resistant tumor is a drug resistant breast cancer tumor.

14. The method of claim 8 further including the act of determining Pgp expression in the patient.

15. The method of claim 8 where the extent of the drug resistant tumor in the patient is determine by region of interest analysis.

16. A method comprising the acts of:
providing a patient with a pharmaceutical composition; and
correlating the severity of a ischemic reperfussion injury caused by the pharmaceutical composition in a tissue of the patient with an amount of ^{99m}Tc GLA localized in the injured tissue of the patient.

17. The method of claim 15 where the ischemic reperfusion injury is a myocardial injury.

18. The method of claim 15 where the size of the injury is determined by measuring the extent of the hot spot from of the ^{99m}Tc-GLA localized in the tissue.

19. The method of claim 16 where the size of the injury is determined by measuring the extent of the hot spot from of the ^{99m}Tc-GLA localized in the myocardium.

20. The method of claim 16 further including the act of modifying the pharmaceutical composition to increase the ischemic reperfusion injury.

21. The method comprising:
providing ischemic preconditioning to a patient with a pharmaceutical composition characterized by the method of claim 15.
